(19) United States
(12) Patent Application Publication
(10) Pub. No.: US 2002/0142995 A1
(43) Pub. Date: Oct. 3, 2002

(54) Ammonium salts of hemoglobin allosteric effectors, and uses thereof

(76) Inventors: Yves Claude Nicolau, Newton, MA (US); Jaime E. Lazarte, Needham, MA (US); Dennis R. Alford, Lynn, MA (US)

Correspondence Address: FOLEY, HOAG & ELIOT, LLP, PATENT GROUP, ONE POST OFFICE SQUARE, BOSTON, MA, 02109, US

(21) Appl. No.: 09/920,310

(22) Filed: Aug. 1, 2001

Related U.S. Application Data
(60) Provisional application No. 60/222,066, filed on Aug. 1, 2000.

(51) Int. Cl. A61K 31/663; C07F 9/02

(52) U.S. Cl. 514/102; 558/156

(57) ABSTRACT

The present invention comprises compounds, compositions thereof, and methods capable of delivering a broad range of anionic molecules to the cytoplasm of mammalian cells. In certain embodiments, the present invention relates to compounds, compositions thereof, and methods that enhance the ability of mammalian red blood cells to deliver oxygen, by delivering a ligand for the allosteric site of hemoglobin to the cytoplasm of the red blood cells.

### RELATED APPLICATION INFORMATION

This application claims the benefit of priority under 35 U.S.C. section 119(e) to Provisional Patent Applications No. 60/222,066, filed Aug. 1, 2000. This application is hereby incorporated by reference in its entirety.

### BACKGROUND OF THE INVENTION

I. Ischemia

Ischemic insult, i.e., the localized deficiency of oxygen to an organ or skeletal tissue, is a common and important problem in many clinical conditions. The problem is especially acute in organ transplant operations in which a harvested organ is removed from a body, isolated from a blood source, and thereby deprived of oxygen and nutrients for an extended period of time. Ischemic insult also occurs in certain clinical conditions, such as sickle cell anemia and septic shock, which may result from hypotension or organ dysfunction. Depending on the duration of the insult, the ischemia can disturb cellular metabolism and ion gradients, and ultimately cause irreversible cellular injury and death.

Arguably, heart attacks and stroke are the most widely recognized example of the damage resulting from ischemia. Myocardial ischemia is a condition wherein there is insufficient blood supply to the myocardium (the muscles of the heart) to meet its demand for oxygen. The ultimate result of persistent myocardial ischemia is necrosis or death of a portion of cardiac muscle tissue, known as a myocardial infarct, commonly known as a heart attack.

Insufficient blood supply to the myocardium is generally due to an obstruction or thrombus in an artery supplying blood to the myocardium. Another cause can be atrial fibrillation, wherein the increased heart rate associated with atrial fibrillation increases the work, and hence the blood demand of the myocardium, while the atrial fibrillation at the same time reduces the blood supply.

Whereas stroke is defined as a sudden impairment of body functions caused by a disruption in the supply of blood to the brain. For instance, a stroke occurs when blood supply to the brain is interrupted for any reason, including hemorrhage, low blood pressure, clogging by atherosclerotic plaque, a blood clot, or any particle. Because of the blockage or rupture, part of the brain fails to get the supply of blood and oxygen that it requires. Brain tissue that receives an inadequate supply of blood is said to be ischemic. Deprived of oxygen and nutrients, nerve cells and other cell types within the brain begin to fail, creating an infarct (an area of cell death, or necrosis). As the neurons fail and die, the part of the body controlled by those neurons can no longer function. The devastating effects of ischemia are often permanent because brain tissue has very limited repair capabilities and lost neurons are typically not regenerated.

Cerebral ischemia may be incomplete (blood flow is reduced but not entirely cut off), complete (total loss of tissue perfusion), transient or permanent. If ischemia is incomplete and persists for no more than ten to fifteen minutes, neural death may not occur. More prolonged or complete -ischemia results in infarction. Depending on the site and extent of the infarction, mild to severe neurological disability or death will follow.

To a modest extent, the brain is protected against cerebral ischemia by compensatory mechanisms, including collateral circulation (overlapping local blood supplies), and arteriolar auto-regulation (local smooth muscle control of blood flow in the smallest arterial channels). If compensatory mechanisms operate efficiently, slightly diminished cerebral blood flow produces neither tissue ischemia nor abnormal signs and symptoms. Usually, such mechanisms must act within minutes to restore blood flow if permanent infarction damage is to be avoided or reduced. Arteriolar auto-regulation works by shunting blood from noncritical regions to infarct zones.

Even in the face of systemic hypotension, auto-regulation may be sufficient to adjust the circulation and thereby preserve the vitality and function of brain or heart tissue. Alternatively, ischemia may be sufficiently prolonged and compensatory mechanisms sufficiently inadequate that a catastrophic stroke or heart attack results.

Ischemia is also associated with various clinical conditions, such as septic shock. Septic shock as a result of hypotension and organ dysfunction in response to infectious sepsis is a major cause of death. The manifestations of sepsis include those related to the systemic response to infection (tachycardia, tachypnea alterations in temperature and leukocytosis) and those related to organ-system dysfunction (cardiovascular, respiratory, renal, hepatic and hematologic abnormalities). Furthermore, the lipopolysaccharide (LPS) of gram-negative bacteria is considered to be the most important exogenous mediator of acute inflammatory response to septic shock. The LPS or endotoxin released from the outer membrane of gram-negative bacteria results in the release of cytokines and other cellular mediators, including tumor necrosis factor alpha (TNF alpha), interleukin-1 (Il-1), interleukin-6 (Il-6) and thromboxane A2. Extreme levels of these mediators are known to trigger many pathological events, including fever, shock, and intravascular coagulation, leading to ischemia and organ failure.

II. Hemoglobin

Hemoglobin is a tetrameric protein which delivers oxygen via an allosteric mechanism. Oxygen binds to the four hemes of the hemoglobin molecule. Each heme contains porphyrin and iron in the ferrous state. The ferrous iron-oxygen bond is readily reversible. Binding of the first oxygen to a heme releases much greater energy than binding of the second oxygen molecule, binding of the third oxygen releases even less energy, and binding of the fourth oxygen releases the least energy.

In blood, hemoglobin is in equilibrium between two allosteric structures. In the “T” (for tense) state, hemoglobin is deoxygenated. In the “R” (for relaxed) state, hemoglobin is oxygenated. An oxygen equilibrium curve can be scanned to observe the affinity and degree of cooperativity (allosteric action) of hemoglobin. In the scan, the Y-axis plots the percent of hemoglobin oxygenation and the X-axis plots the partial pressure of oxygen in millimeters of mercury (mm Hg). If a horizontal line is drawn from the 50% oxygen saturation point to the scanned curve and a vertical line is drawn from the intersection point of the horizontal line with the curve to the partial pressure X-axis, a value commonly known as the P₅₀ is determined (i.e., this is the pressure in mm Hg when the scanned hemoglobin sample is 50% saturated with oxygen). Under physiological conditions (i.e., 37 C., pH=7.4, and partial carbon dioxide pressure of 40 mm Hg), the P₅₀ value for normal adult hemoglobin (HbA) is around 26.5 mm Hg. If a lower than normal P₅₀ value is obtained for the hemoglobin being tested, the scanned curve is considered to be “left-shifted” and the presence of high oxygen-affinity hemoglobin is indicated. Conversely, if a higher than normal P₅₀ value is obtained for the hemoglobin being tested, the scanned curve is considered to be “right-shifted”, indicating the presence of low oxygen-affinity hemoglobin.

It has been proposed that influencing the allosteric equilibrium of hemoglobin is a viable avenue of attack for treating diseases. The conversion of hemoglobin to a high affinity state is generally regarded to be beneficial in resolving problems with (deoxy)hemoglobin-S (i.e., sickle cell anemia). The conversion of hemoglobin to a low affinity state is believed to have general utility in a variety of disease states where tissues suffer from low oxygen tension, such as ischemia and radio sensitization of tumors. Several synthetic compounds have been identified which have utility in the allosteric regulation of hemoglobin and other proteins. For example, several new compounds and methods for treating sickle cell anemia which involve the allosteric regulation of hemoglobin are reported in U.S. Pat. No. 4,699,926 to Abraham et al., U.S. Pat. No. 4,731,381 to Abraham et al., U.S. Pat. No. 4,731,473 to Abraham et al., U.S. Pat. No. 4,751,244 to Abraham et al., and U.S. Pat. No. 4,887,995 to Abraham et al. Furthermore, in both Perutz, “Mechanisms of Cooperativity and allosteric Regulation in Proteins”, Quarterly Reviews of Biophysics 22, 2 (1989), pp. 163-164, and Lalezari et al., “LR16, a compound with potent effects on the oxygen affinity of hemoglobin, on blood cholesterol, and on low density lipoprotein”, Proc. Natl. Acad. Sci., USA 85 (1988), pp. 6117-6121, compounds which are effective allosteric hemoglobin modifiers are discussed. In addition, Perutz et al. has shown that a known antihyperlipoproteinemia drug, bezafibrate, is capable of lowering the affinity of hemoglobin for oxygen (See “Bezafibrate lowers oxygen affinity of hemoglobin”, Lancet 1983, 881).

Human normal adult hemoglobin (“HbA”) is a tetrameric protein containing two alpha chains having 141 amino acid residues each and two beta chains having 146 amino acid residues each, and also bearing prosthetic groups known as hemes. The erythrocytes help maintain hemoglobin in its reduced, functional form. The heme-iron atom is susceptible to oxidation, but may be reduced again by one of two systems within the erythrocyte, the cytochrome b5, and glutathione reduction systems.

Hemoglobin is able to alter its oxygen affinity, thereby increasing the efficiency of oxygen transport in the body due to its dependence on 2,3-DPG, an allosteric regulator. 2,3-DPG is present within erythrocytes at a concentration that facilitates hemoglobin to release bound oxygen to tissues. Naturally-occurring hemoglobin includes any hemoglobin identical to hemoglobin naturally existing within a cell. Naturally-occurring hemoglobin is predominantly wild-type hemoglobin, but also includes naturally-occurring mutant hemoglobin. Wild-type hemoglobin is hemoglobin most commonly found within natural cells. Wild-type human hemoglobin includes hemoglobin A, the normal adult human hemoglobin having two alpha—and two beta-globin chains. Mutant hemoglobin has an amino-acid sequence that differs from the amino-acid sequence of wild-type hemoglobin as a result of a mutation, such as a substitution, addition or deletion of at least one amino acid. Adult human mutant hemoglobin has an amino-acid sequence that differs from the amino-acid sequence of hemoglobin A. Naturally-occurring mutant hemoglobin has an amino-acid sequence that has not been modified by humans. The naturally-occurring hemoglobin of the present invention is not limited by the methods by which it is produced. Such methods typically include, for example, erythrocytolysis and purification, recombinant production, and protein synthesis.

It is known that hemoglobin specifically binds small polyanionic molecules, especially 2,3-diphosphoglycerate (DPG) and adenosine triphosphate (ATP), present in the mammalian red cell (Benesch and Benesch, Nature, Vol. 221, p. 618, 1969). This binding site is located at the centre of the tetrameric structure of hemoglobin (Arnone, A., Nature, Vol. 237, p. 146, 1972). The binding of these polyanionic molecules is important in regulating the oxygen-binding affinity of hemoglobin since it allosterically affects the conformation of hemoglobin leading to a decrease in oxygen affinity (Benesch and Benesch, Biochem. Biophys. Res. Comm., Vol 26, p. 162, 1967). Conversely, the binding of oxygen allosterically reduces the affinity of hemoglobin for the polyanion. (Oxy)hemoglobin therefore binds DPG and ATP weakly. This is shown, for example, by studies of spin-labeled ATP binding to oxy- and deoxyhemoglobin as described by Ogata and McConnell (Ann. N.Y. Acad. Sc., Vol. 222, p. 56, 1973). In order to exploit the polyanion-binding specificity of hemoglobin, or indeed to perform any adjustment of its oxygen-binding affinity by chemically modifying the polyanion binding site, it has been necessary in the prior art that hemoglobin be deoxygenated. However, hemoglobin as it exists in solutions, or mixtures exposed to air, is in its oxy state, i.e., (oxy)hemoglobin. In fact it is difficult to maintain hemoglobin solutions in the deoxy state, (deoxy)hemoglobin, throughout a chromatographic procedure. Because of these difficulties, the technique of affinity chromatography has not been used in the prior art to purify hemoglobin.

Hemoglobin has also been administered as a pretreatment to patients receiving chemotherapeutic agents or radiation for the treatment of tumors (U.S. Pat. No. 5,428,007; WO 92/20368; WO 92/20369), for prophylaxis or treatment of systemic hypotension or septic shock induced by internal nitric oxide production (U.S. Pat. No. 5,296,466), during the perioperative period or during surgery in a method for maintaining a steady-state hemoglobin concentration in a patient (WO 95/03068), and as part of a perioperative hemodilution procedure used prior to surgery in an autologous blood use method (U.S. Pat. Nos. 5,344,393 and 5,451,205). When a patient suffers a trauma (i.e., a wound or injury) resulting, for example, from surgery, an invasive medical procedure, or an accident, the trauma disturbs the patient's homeostasis. The patient's body biologically reacts to the trauma to restore homeostasis. This reaction is referred to herein as a naturally occurring stress response. If the body's stress response is inadequate or if it occurs well after the trauma is suffered, the patient is more prone to develop disorders.

III. Reduction of the Oxygen-Affinity of Hemoglobin

The major function of erythrocytes consists in the transport of molecular oxygen from the lungs to the peripheral tissues. The erythrocytes contain a high concentration of hemoglobin (30 pg per cell=35.5 g/100 ml cells) which forms a reversible adduct with O₂. The O₂-partial pressure in the lung is about.100 mm Hg, in the capillary system is about.70 mm Hg, against which O₂ must be dissociated from the oxygenated hemoglobin. Under physiological conditions, only about 25% of the oxygenated hemoglobin may be deoxygenated; about.75% is carried back to the lungs with the venous blood. Thus, the major fraction of the hemoglobin-O₂ adduct is not used for the O₂ transport.

Interactions of hemoglobin with allosteric effectors enable an adaptation to the physiological requirement of maximum O₂ release from the hemoglobin-O₂ adduct with simultaneous conservation of the highest possible O₂ partial pressure in the capillary system. 2,3-Diphosphoglycerate increases the half-saturation pressure of stripped hemoglobin at pH 7.4 from P(O₂) (½)=9.3 mm Hg (37 C.), and 4.3 mm Hg (25 C.) to P(O₂) (½)=23.7 mm Hg (37C.) and 12.0 mm Hg (25 C.), respectively (Imai, K. and Yonetani, T. (1975), J. Biol. Chem. 250, 1093-1098). A significantly stronger decrease of the O₂ affinity, i.e., enhancement of the O₂ half-saturation pressure has been achieved for stripped hemoglobin by binding of inositol hexaphosphate (phytic acid; IHP) (Ruckpaul, K. et al. (1971) Biochim. Biophys. Acta 236, 211-221) isolated from vegetal tissues. Binding of IHP to hemoglobin increases the O₂ half-saturation pressure to P(O₂) (½)=96.4 mm Hg (37 C.), and P(O₂) (1/2)=48.4 mm Hg (25C.) respectively. IHP, like 2,3-diphosphoglycerate and other polyphosphates cannot penetrate the erythrocyte membrane.

Furthermore, the depletion of DPG and ATP in stored red cells leads to a progressive increase of the oxygen affinity of hemoglobin contained therein (Balcerzak, S. et al. (1972) Adv. Exp. Med. Biol. 28, 453-447). The O₂-binding isotherms are measured in the absence of CO₂ and at constant pH (pH 7.4) in order to preclude influences of these allosteric effectors on the half-saturation pressure. The end point of the progressive polyphosphate depletion is defined by P(O₂) (½)=4.2 mm Hg, which is the half-saturation pressure of totally phosphate-free (stripped) hemoglobin; the starting point, i.e., P(O₂) (½) of fresh erythrocytes, depends on the composition of the suspending medium. From these polyphosphate depletion curves a new functional parameter of stored erythrocytes can be determined, the so-called half-life time of intra-erythrocytic polyphosphate: 9 d (days) in isotonic 0.1 M bis-Tris buffer pH 7.4; and 12 d (days) in acid-citrate-dextrose conservation (ACD) solution.

Several years ago, it was discovered that the antilipidemic drug clofibric acid lowered the oxygen affinity of hemoglobin solutions (Abraham et al., J. Med. Chem. 25, 1015 (1982), and Abraham et al., Proc. Natl. Acad. Sci. USA 80, 324 (1983)). Bezafibrate, another antilipidemic drug, was later found to be much more effective in lowering the oxygen affinity of hemoglobin solutions and suspensions of fresh, intact red cells (Perutz et al., Lancet, 881, Oct. 15, 1983). Subsequently, X-ray crystallographic studies have demonstrated that clofibric acid and bezafibrate bind to the same sites in the central water cavity of deoxyhemoglobin, and that one bezafibrate molecule will span the sites occupied by two clofibric acid molecules. Bezafibrate and clofibric acid act by stabilizing the deoxy structure of hemoglobin, shifting the allosteric equilibrium toward the low affinity deoxy form. Bezafibrate and clofibric acid do not bind in any specific manner to either oxy- or carbonmonoxyhemoglobin.

In more recent investigations, a series of urea derivatives [2-[4-[[(arylamino)carbonyl]amino]phenoxy]-2-methylpropionic acids] was discovered that has greater allosteric potency than bezafibrate at stabilizing the deoxy structure of hemoglobin and shifting the allosteric equilibrium toward the low oxygen affinity form (Lalezari, Proc. Natl. Acad. Sci. USA 85, 6117 (1988)).

Drugs which can allosterically modify hemoglobin toward a lower oxygen affinity state hold potential for many clinical applications, such as for the treatment of ischemia, shock, and polycythemia, and as radiosensitizing agents. Unfortunately, the effects of bezafibrate and the urea derivatives discussed above have been found to be significantly inhibited by serum albumin, the major protein in blood serum (Lalezari et al., Biochemistry, 29, 1515 (1990)). Therefore, the clinical usefulness of these drugs is seriously undermined because in whole blood and in the body, the drugs would be bound by serum albumin instead of reaching the red cells, crossing the red cell membrane, and interacting with hemoglobin protein molecule to produce the desired effect.

There has been considerable interest in medicine, the military health services, and the pharmaceutical industry in finding methods to increase blood storage life; to discover radio sensitization agents; and to develop new blood substitutes. In all these instances, the availability of either autologous blood or recombinant Hb solutions is of major interest, provided the oxygen affinity can be decreased to enhance oxygen delivery to the tissues.

2,3-Diphosphoglycerate (2,3-DPG) is the normal physiological ligand for the allosteric site on hemoglobin. However, phosphorylated inositols are found in the erythrocytes of birds and reptiles. Specifically, inositol hexaphosphate (IHP), as known as phytic acid, displaces hemoglobin-bound 2,3-DPG, binding to the allosteric site with one-thousand times greater affinity. Unfortunately, IHP is unable to pass unassisted across the erythrocyte membrane.

IV. Enhanced Oxygen Delivery in Mammals

The therapy of oxygen deficiencies requires the knowledge of parameters which characterize both the O₂ transport capacity and the O₂ release capacity of human RBCs. The parameters of the O₂ transport capacity, i.e., Hb concentration, the number of RBCs, and hemocrit, are commonly used in clinical diagnosis. However, the equally important parameters of the O₂ release capacity, i.e., O₂ half-saturation pressure of Hb and RBCs, and the amounts of high and low oxygen affinity hemoglobins in RBCs, are not routinely determined and were not given serious consideration until pioneering work by Gerosonde and Nicolau (Blut, 1979, 39, 1-7).

In the 1980s, Nicolau et al. (J. Appl. Physiol. 58:1810-1817 (1985); “PHYTIC ACID: Chemsitry and Applications”; Graf, E., Ed.; Pilatus Press, Minneapolis, Minn., USA; 1986; and Proc. Natl. Acad. Sci. USA 1987, 84, 6894-6898) reported that the encapsulation in red blood cells (RBCs) of IHP, via a technique of controlled lysis and resealing, results in a significant decrease in the hemoglobin affinity for oxygen. The procedure yielded RBCs with unchanged life spans, normal ATP and K+ levels, and normal rheological competence. Enhancement of the O₂-release capacity of these cells brought about significant physiological effects in piglets: 1) reduced cardiac output, linearly dependent on the P50 value of the RBCs; 2) increased arteriovenous difference; and 3) improved tissue oxygenation. Long term experiments showed that in piglets the high P50 value of IHP-RBCs was maintained over the entire life spans of the RBCs.

More recently, Nicolau et al. (TRANSFUSION 1995, 35, 478-486; and U.S. Pat. No. 5,612,207) reported the use of a large-volume, continuous-flow electroporation system for the encapsulating IHP in human RBCs. These modified RBCs possess P₅₀ values of approximately 50 torr, roughly twice that of unmodified human RBCs. Additionally, 85% of the RBCs survived the electroporation process, displaying hematologic indices nearly identical to those of unmodified RBCs. Nicolau's electroporation system processes one unit of blood every ninety minutes.

V. Specific Clinical Applications of Enhanced Oxygen Delivery

There are numerous clinical conditions that would benefit from treatments that would increase tissue delivery of oxygen bound to hemoglobin. For example, the leading cause of death in the United States today is cardiovascular disease. The acute symptoms and pathology of many cardiovascular diseases, including congestive heart failure, myocardial infarction, stroke, intermittent claudication, and sickle cell anemia, result from an insufficient supply of oxygen in fluids that bathe the tissues. Likewise, the acute loss of blood following hemorrhage, traumatic injury, or surgery results in decreased oxygen supply to vital organs. Without oxygen, tissues at sites distal to the heart, and even the heart itself, cannot produce enough energy to sustain their normal functions. The result of oxygen deprivation is tissue death and organ failure.

Although the attention of the American public has long been focused on the preventive measures required to alleviate heart disease, such as exercise, appropriate dietary habits, and moderation in alcohol consumption, deaths continue to occur at an alarming rate. Since death results from oxygen deprivation, which in turn results in tissue destruction and/or organ dysfunction, one approach to alleviate the life-threatening consequences of cardiovascular disease is to increase oxygenation of tissues during acute stress. The same approach is also appropriate for persons suffering from blood loss or chronic hypoxic disorders, such as congestive heart failure.

Another condition which could benefit from an increase in the delivery of oxygen to the tissues is anemia. A significant portion of hospital patients experience anemia or a low “crit” caused by an insufficient quantity of red blood cells or hemoglobin in their blood. This leads to inadequate oxygenation of their tissues and subsequent complications. Typically, a physician can temporarily correct this condition by transfusing the patient with units of packed red blood cells.

Enhanced blood oxygenation may also reduce the number of heterologous transfusions and allow use of autologous transfusions in more case. The current method for treatment of anemia or replacement of blood loss is transfusion of whole human blood. It is estimated that three to four million patients receive transfusions in the U.S. each year for surgical or medical needs. In situations where there is more time it is advantageous to completely avoid the use of donor or heterologous blood and instead use autologous blood.

Often the amount of blood which can be drawn and stored prior to surgery limits the use of autologous blood. Typically, a surgical patient does not have enough time to donate a sufficient quantity of blood prior to surgery. A surgeon would like to have several units of blood available. As each unit requires a period of several weeks between donations and can not be done less than two weeks prior to surgery, it is often impossible to sequester an adequate supply of blood. By processing autologous blood with IHP, less blood is required and it becomes possible to completely avoid the transfusion of heterologous blood.

Because IHP-treated RBCs may release up to 2-3 times as much oxygen as untreated red cells, in many cases, a physician will need to transfuse fewer units of IHP-treaded red cells. This exposes the patient to less heterologous blood, decreases the extent of exposure to vital diseases from blood donors and minimizes immune function disturbances secondary to transfusions. The ability to infuse more efficient red blood cells is also advantageous when the patients blood volume is excessive. In more severe cases, where oxygen transport is failing, the ability to improve rapidly a patient's tissue oxygenation is life saving.

Although it is evident that methods of enhancing oxygen delivery to tissues have potential medical applications, currently there are no methods clinically available for increasing tissue delivery of oxygen bound to hemoglobin. Transient, 6 to 12 hour elevations of oxygen deposition have been described in experimental animals using either DPG or molecules that are precursors of DPG. The natural regulation of DPG synthesis in vivo and its relatively short biological half-life, however, limit the DPG concentration and the duration of increased tissue PO₂, and thus limit its therapeutic usefulness.

Additionally, as reported in Genetic Engineering News, Vol. 12, No. 6, Apr. 15, 1992, several groups are attempting to engineer free oxygen-carrying hemoglobin as a replacement for human blood. Recombinant, genetically modified human hemoglobin that does not break down in the body and that can readily release up to 30% of its bound oxygen is currently being tested by Somatogen, Inc., of Boulder Colo. While this product could be useful as a replacement for blood lost in traumatic injury or surgery, it would not be effective to increase PO₂ levels in ischemic tissue, since its oxygen release capacity is equivalent to that of natural hemoglobin (27-30%). As are all recombinant products, this synthetic hemoglobin is also likely to be a costly therapeutic.

Synthetic human hemoglobin has also been produced in neonatal pigs by injection of human genes that control hemoglobin production. This product may be less expensive product than the Somatogen synthetic hemoglobin, but it does not solve problems with oxygen affinity and breakdown of hemoglobin in the body.

### SUMMARY OF THE INVENTION

The present invention relates to compositions, and methods of use thereof, consisting essentially of aliphatic ammonium cations (preferably water-soluble), and an allosteric effector, i.e., ligand for the allosteric site, of hemoglobin, e.g., inositol hexaphosphate (IHP).

The aliphatic ammonium cation is substituted with one or more times with aliphatic groups, which can be the same or different. In certain embodiments, the aliphatic ammonium cation is a primary ammonium cation represented by the general formula NH₃(R), wherein R is an aliphatic group, preferably an alkyl, more preferably a lower alkyl, i.e., a C₁-C₆ alkyl, and even more preferably a C₃-C₆ cycloalkyl. In certain preferred embodiments, the ammonium cation is preferably derived from cyclic amines.

In certain embodiments, the present invention relates to compounds, and compositions thereof, that deliver into erythrocytes allosteric effectors of hemoglobin ex vivo, for lowering the oxygen affinity of hemoglobin in red blood cell suspensions and whole blood. It is an object of this invention to provide methods for delivering into erythrocytes allosteric effectors of hemoglobin in whole blood and, utilizing compounds, or compositions thereof, that do not lose their effectiveness in the presence of normal concentrations of the remaining components of whole blood.

In certain embodiments, the present invention relates to a method of treating a subject for any one or more diseases where an increase in oxygen delivery of hemoglobin would be of benefit comprising the steps of treating red blood cells or whole blood ex vivo with one or more compounds or compositions of the present invention, followed by suitably purifying said red blood cells or whole blood, and administering the thus prepared red blood cells or whole blood to said subject. By ‘suitably purifying’ it is meant a method of washing and separating, for example by centrifugation, the red blood cell- or whole blood-allosteric effector suspension and discarding the supernatant until no non-encapsulated allosteric effector can be detected. An exemplary method is presented in detail by Nicolau et al. in U.S. Pat. No. 5,612,207, which is incorporated by reference herein.

Ligands for the allosteric site of hemoglobin interact with the hemoglobin molecule and impact its ability to bind oxygen. This invention is particularly concerned with the delivery into erythrocytes of ligands for the hemoglobin allosteric site, causing oxygen to be bound relatively less tightly to hemoglobin, such that oxygen is off-loaded from the hemoglobin molecule more easily.

The process of allosterically modifying hemoglobin towards a lower oxygen affinity state in whole blood may be used in a wide variety of applications, including treatments for ischemia, heart disease, wound healing, radiation therapy of cancer, and adult respiratory distress syndrome (ARDS). Furthermore, a decrease in the oxygen affinity of hemoglobin in whole blood will extend its useful shelf-life vis à vis transfusions, and/or restore the oxygen carrying capacity of aged blood.

### BRIEF DESCRIPTION OF THE FIGURES

FIG. 1 tabulates the names or structures of ammonium salts of inositol hexaphosphate and 2,3-diphospho-D-glyceric acid and the corresponding abbreviations used herein.

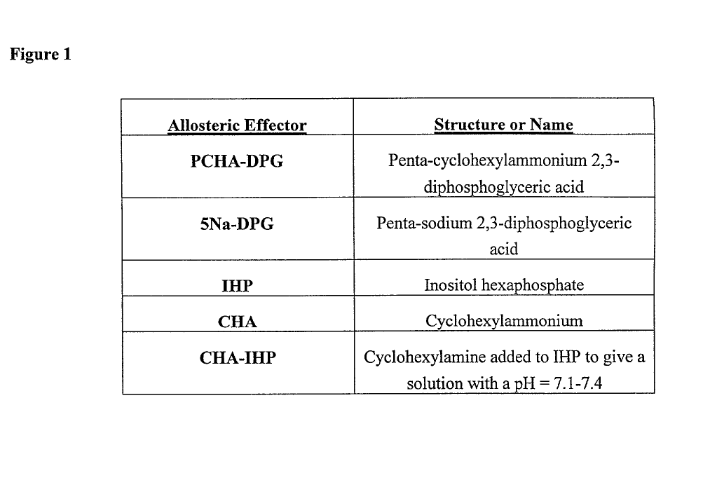

FIG. 2 tabulates the P₅₀ values at various osmolarities of whole blood and free hemoglobin that has been pre-incubated with various ammonium salts of inositol hexaphosphate.

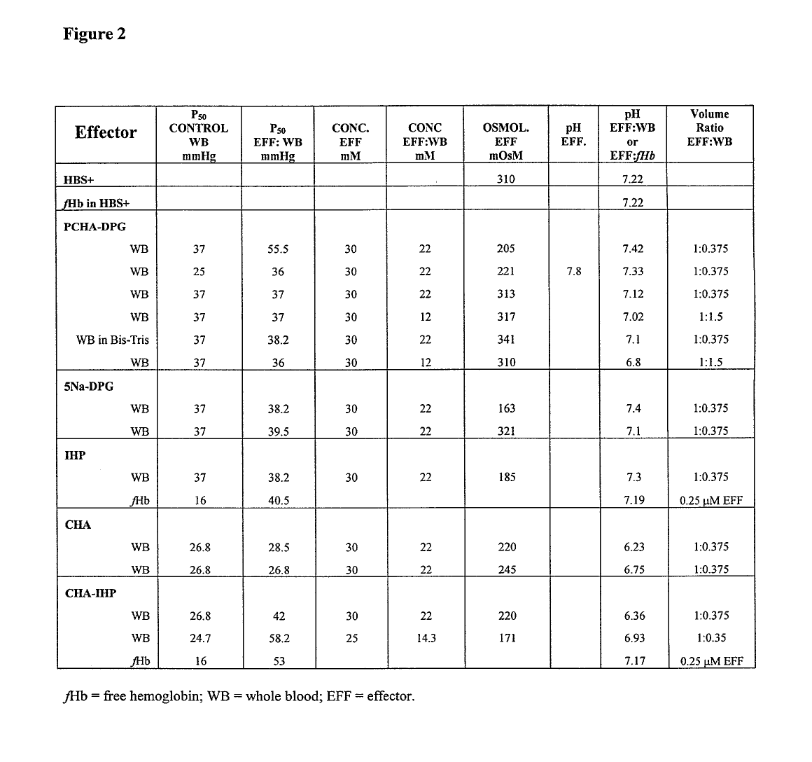

FIG. 3 tabulates the P₅₀ values of human and goldfish whole blood controls, human and goldfish free hemoglobin controls, and goldfish free hemoglobin that has been pre-incubated with various allosteric effectors and an ammonium salt of one of them.

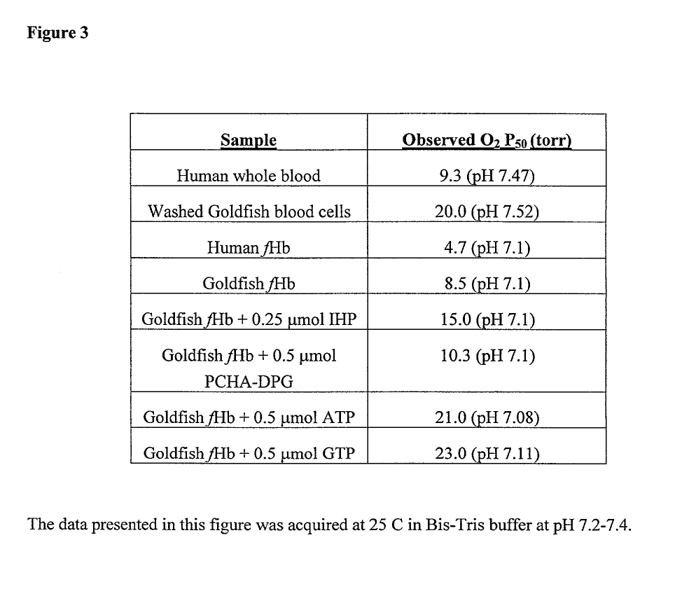

FIG. 4 depicts oxygen dissociation curves of human whole blood (pH 7.47; P₅₀=9.3) and washed goldfish blood (pH 7.52; P₅₀=20.0).

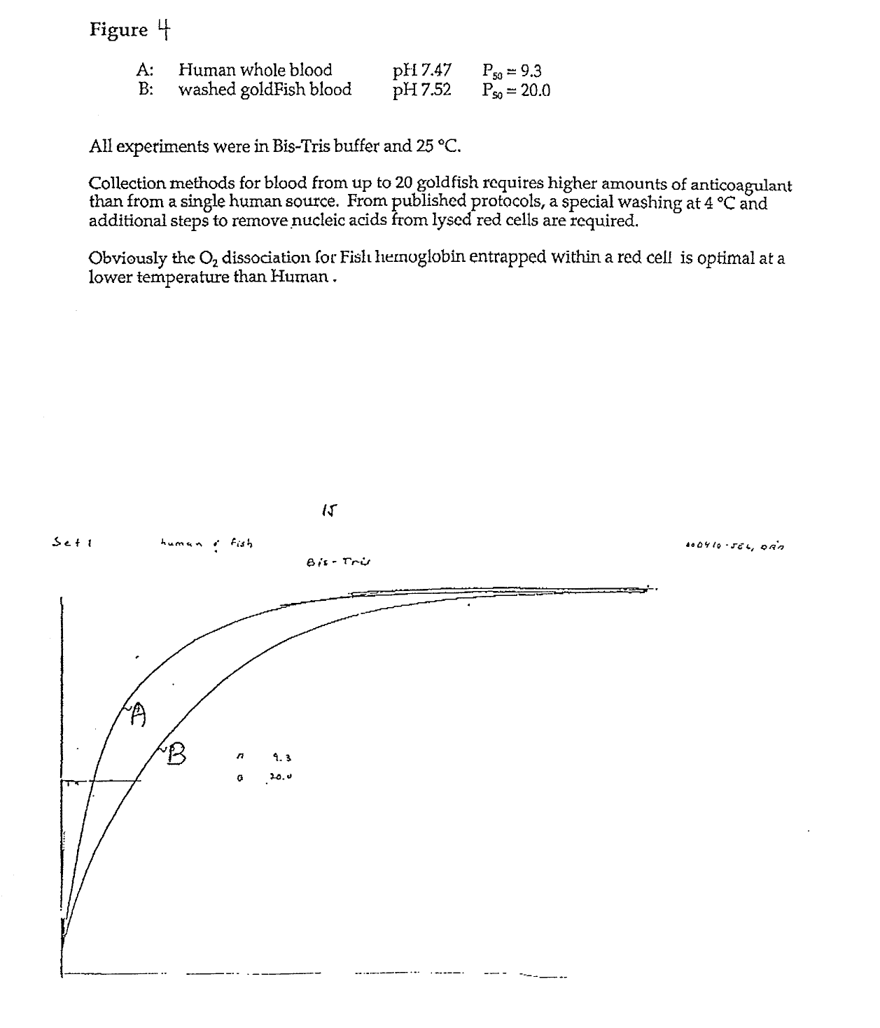

FIG. 5 depicts oxygen dissociation curves of human free hemoglobin (pH 7.1; P₅₀4.7), goldfish free hemoglobin (pH 7.1; P₅₀=8.5), and goldfish free hemoglobin+0.25 μmol IHP (pH 7.1; P₅₀=15.0).

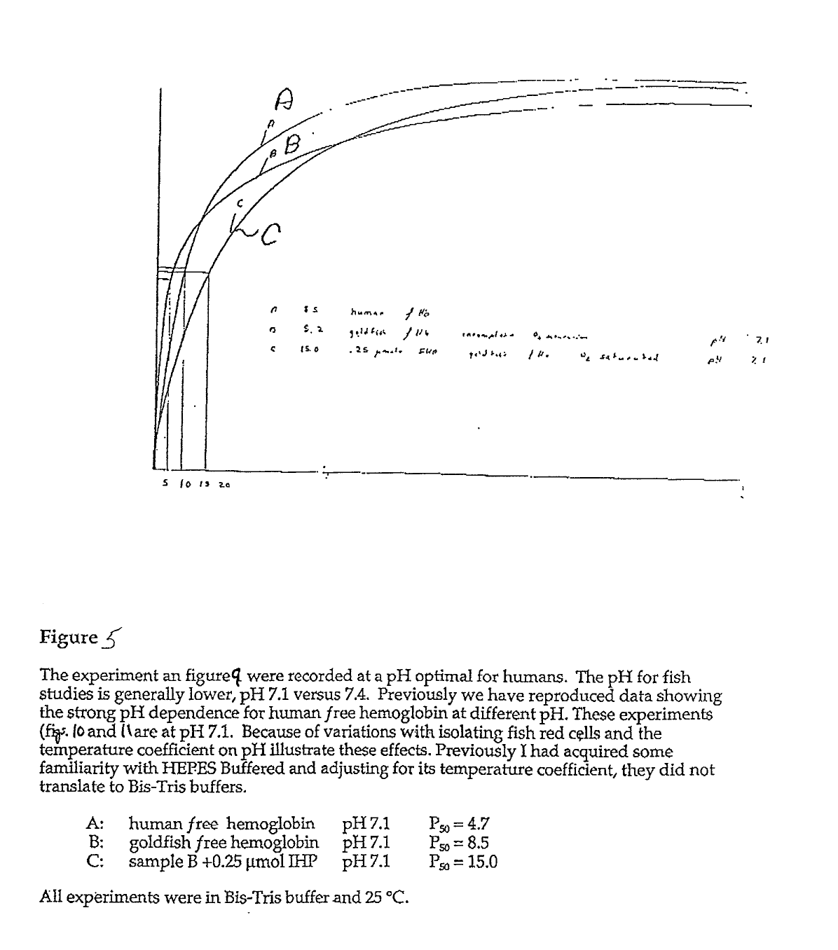

FIG. 6 depicts oxygen dissociation curves of goldfish free hemoglobin+0.5 μmol PCHA-DPG (pH 7.1; P₅₀=10.3), goldfish free hemoglobin+0.5 μmol ATP (pH 7.08; P₅₀=21.0), and goldfish free hemoglobin+0.5 μmol GTP (pH 7.11; P₅₀=23.0).

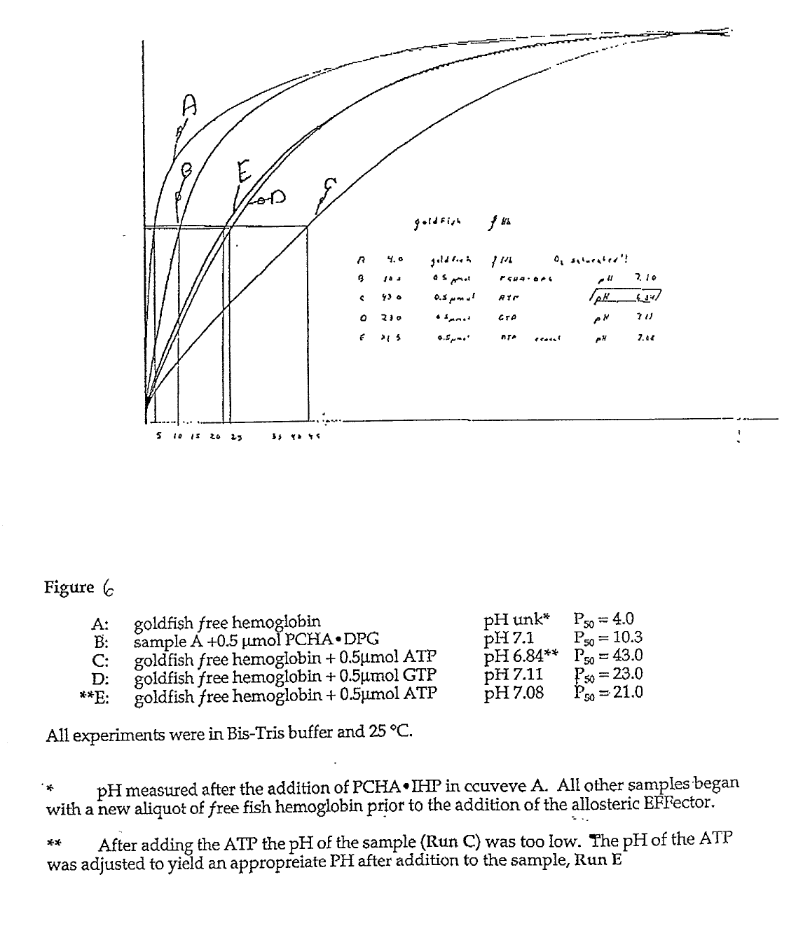

FIG. 7 depicts oxygen dissociation curves of whole blood treated with a solution of pentacyclohexylammonium-2,3 diphosphogliceric acid (PCHA-DPG) and the sodium salt of DPG (PNa-DPG).

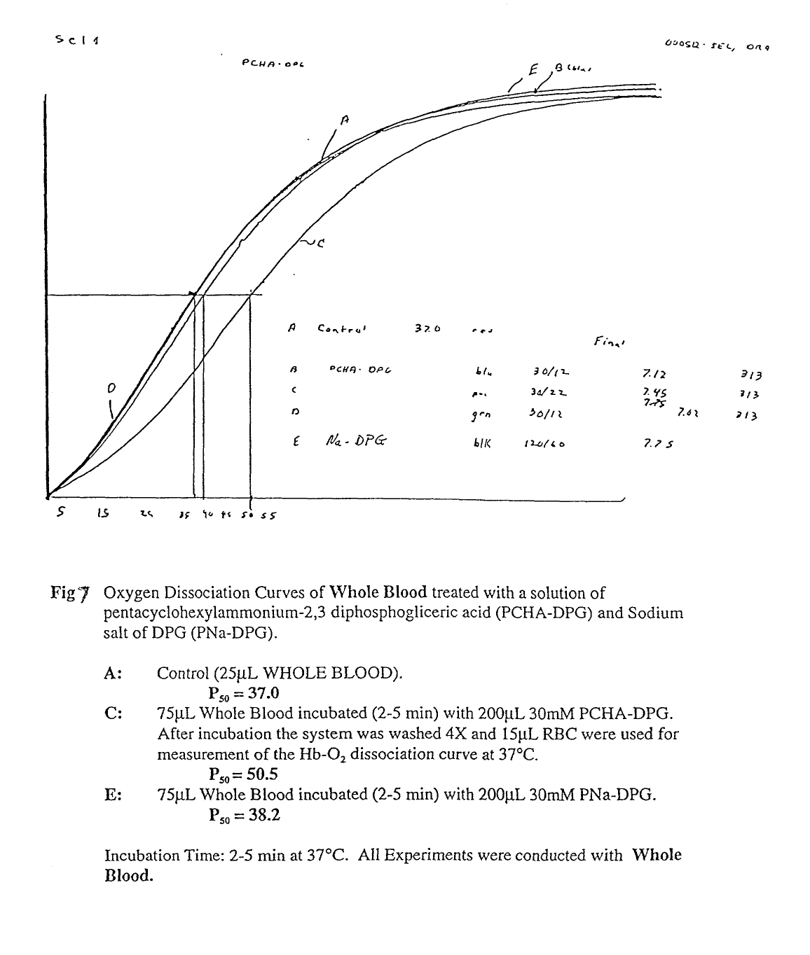

A: Control (25 μL WHOLE BLOOD). Observed P₅₀=37.0

C: 75 μL Whole blood incubated (2-5 min) with 200 μL 30 mM PCHA-DPG. After incubation the system was washed 4× and 15 μL RBC were used for measurement of the Hb-O₂ dissociation curve at 37° C. Observed P₅₀=50.5

E: 75 μL Whole blood incubated (2-5 min) with 200 μL 30 mM PNa-DPG. Observed P₅₀=38.2 Incubation Time: 2-5 min at 37° C. All experiments were conducted with whole blood.

FIG. 8 depicts oxygen dissociation curves of whole blood treated with a solution of pentacyclohexylammonium-2,3 disphosphogliceric acid acid (PCHA-DPG).

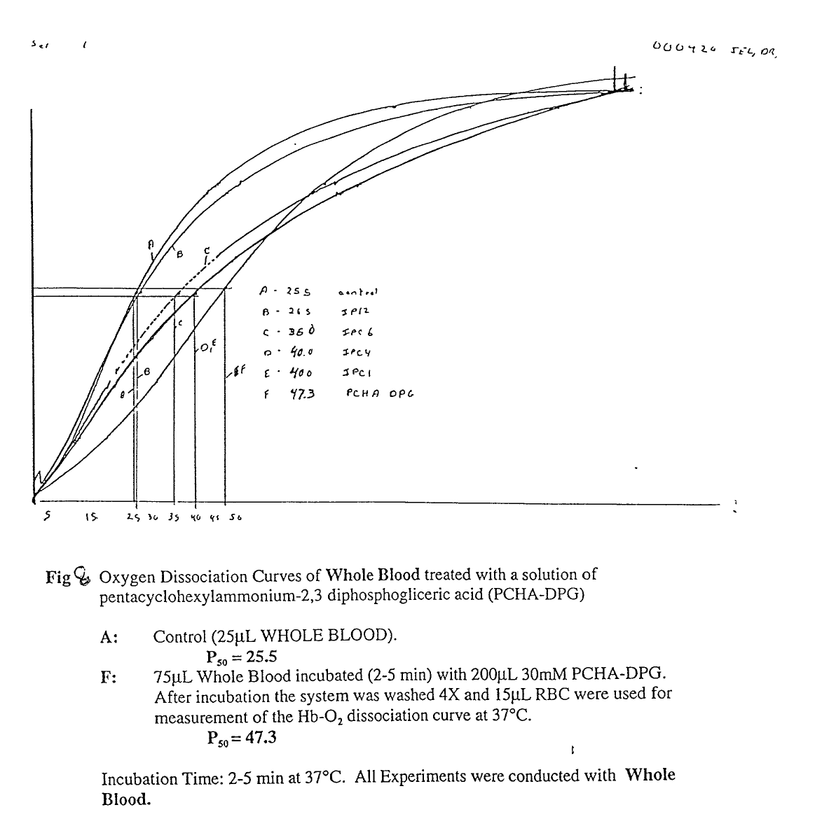

A: Control (25 μL WHOLE BLOOD). Observed P₅₀=25.5

F: 75 μL Whole blood incubated (2-5 min) with 200 μL 30 mM PCHA-DPG. After incubation the system was washed 4× and 15 μL RBC were used for measurement of the Hb-O₂ dissociation curve at 37° C. Observed P₅₀=47.3 Incubation Time: 2-5 min at 37° C. All Experiments were conducted with whole blood.

FIG. 9 depicts oxygen dissociation curves of whole blood treated with a solution of pentacyclohexylammonium-2,3 diphosphogliceric acid (PCHA-DPG).

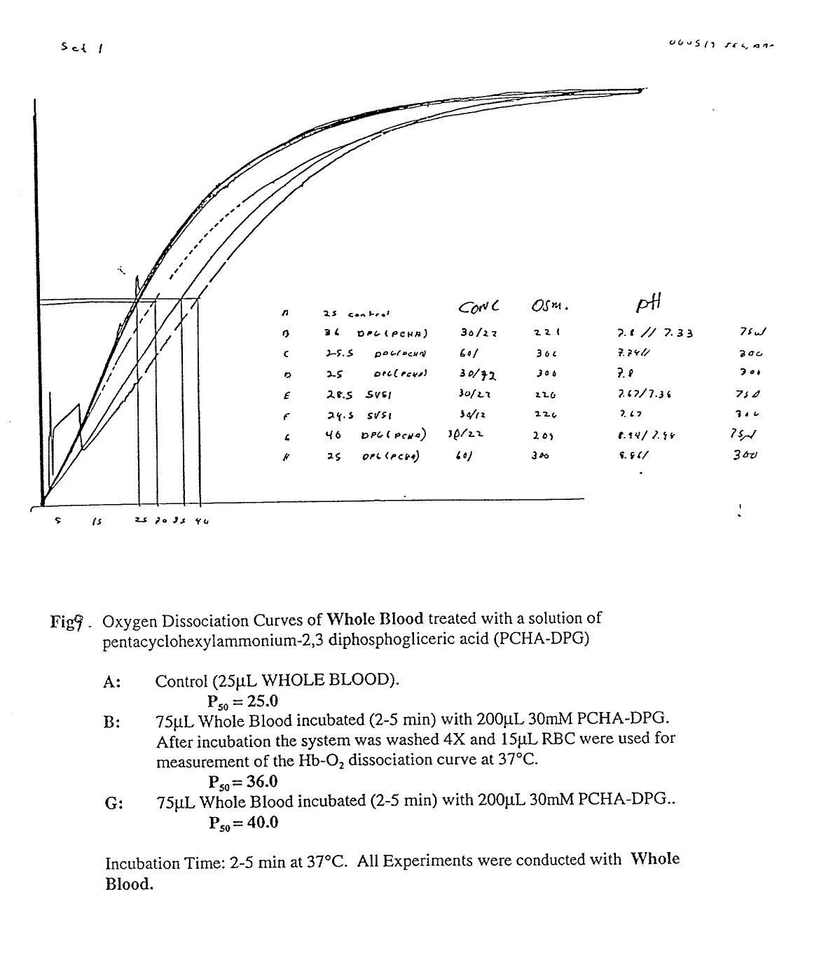

A: Control (25 μL WHOLE BLOOD). Observed P₅₀=25.0

B: 75 μL Whole blood incubated (2-5 min) with 200 μL 30 mM PCHA-DPG. After incubation the system was washed 4× and 15 μL RBC were used for measurement of the Hb-O₂ dissociation curve at 37° C. Observed P₅₀=36.0

G: 75 μL Whole blood incubated (2-5 min) with 200 μL 30 mM PNa-DPG. Observed P₅₀=38.2 Incubation Time: 2-5 min at 37° C. All experiments were conducted with whole blood.

FIG. 10 depicts oxygen dissociation curves of whole blood treated with a solution of Sodium Salts of DPG and IHP.

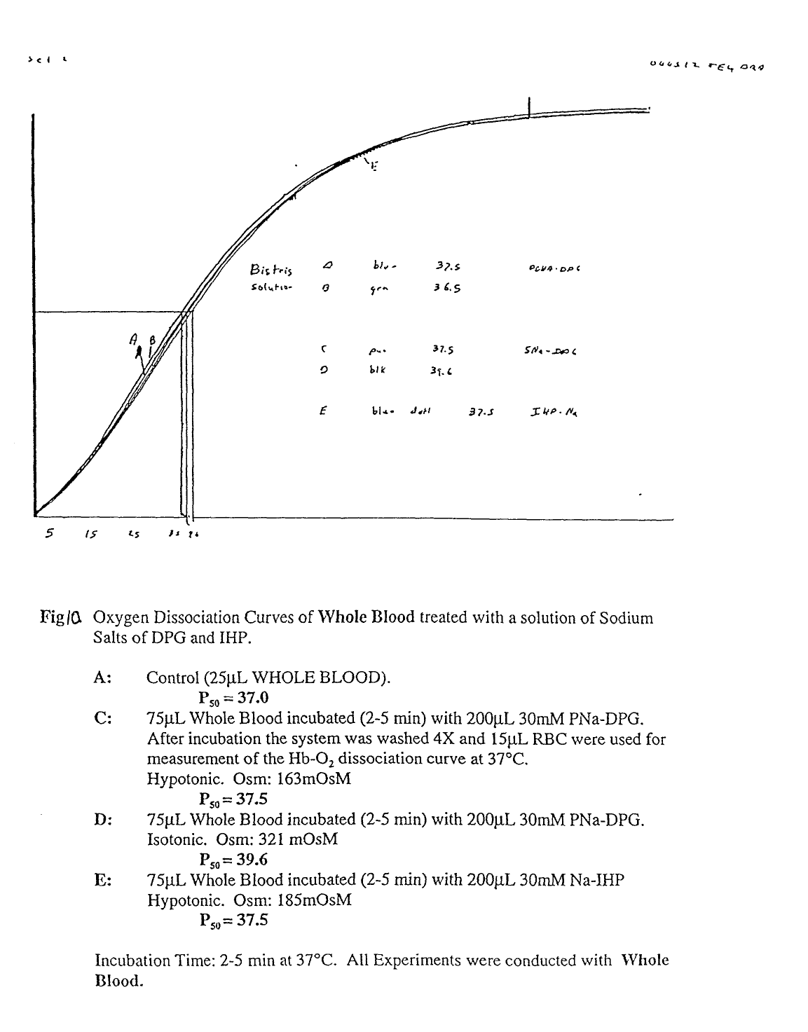

A: Control (25 μL WHOLE BLOOD). Observed P₅₀=37.0

C: 75 μL Whole blood incubated (2-5 min) with 200 μL 30 mM PNa-DPG. After incubation the system was washed 4× and 15 μL RBC were used for measurement of the Hb-O₂ dissociation curve at 37° C. Hypotonic. Osm: 163 mOsM. Observed P₅₀=37.5

D: 75 μL Whole blood incubated (2-5 min) with 200 μL 30 mM PNa-DPG. Isotonic. Osm: 321 mOsM. Observed P₅₀=39.6

E: 75 μL Whole blood incubated (2-5 min) with 200 μL 30 mM Na-IHP. Hypotonic. Osm: 185 mOsM. Observed P₅₀=37.5 Incubation Time: 2-5 min at 37° C. All Experiments were conducted with whole blood.

FIG. 11 depicts oxygen dissociation curves of whole blood treated with a solution of cyclohexylammonium (CHA) and CHA salt of IHP.

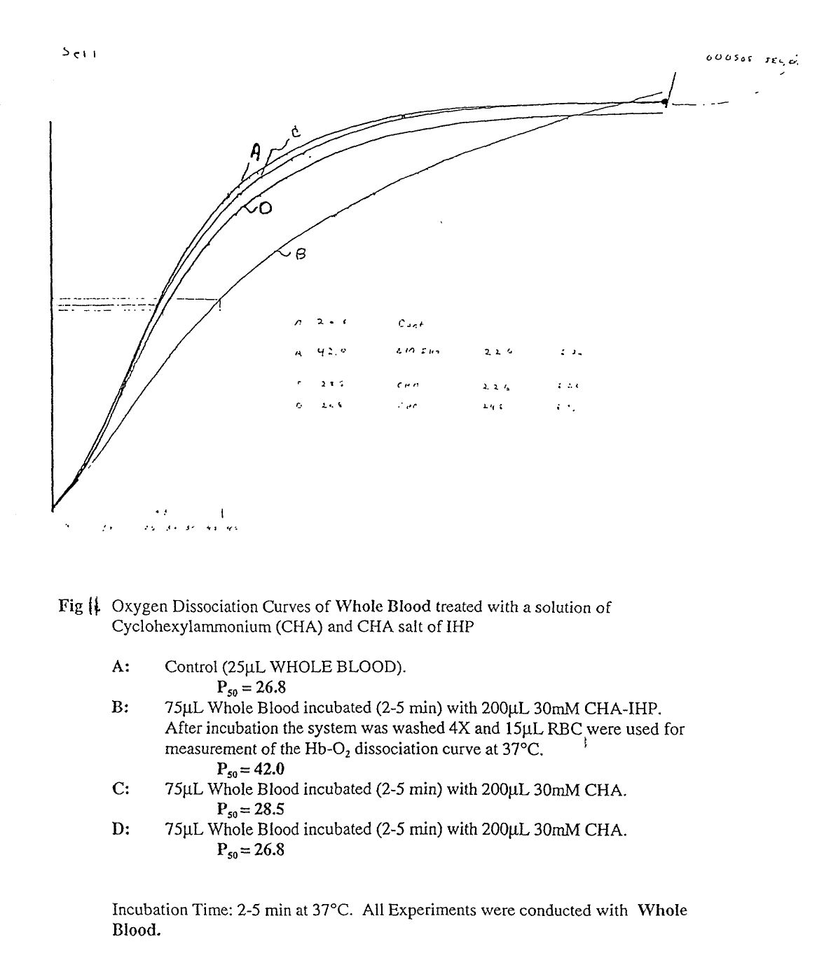

A: Control (25 μL WHOLE BLOOD). Observed P₅₀=26.8

B: 75 μL Whole blood incubated (2-5 min) with 200 μL 30 mM CHA-IHP. After incubation the system was washed 4× and 15 μL RBC were used for measurement of the Hb-O₂ dissociation curve at 37° C. Observed P₅₀=42.0

C: 75 μL Whole blood incubated (2-5 min) with 200 μL 30 mM CHA. Observed P₅₀=28.5

D: 75 μL Whole blood incubated (2-5 min) with 200 μL 30 mM CHA. Observed P₅₀=26.8 Incubation Time: 2-5 min at 37° C. All Experiments were conducted with whole blood.

FIG. 12 depicts oxygen dissociation curves of whole blood treated with a solution of Cyclohexylammonium-Inositol Hexaphosphate (CHA-IHP).

D: Control (25 μL WHOLE BLOOD). Observed P₅₀=24.7

C: 75 μL Whole blood incubated (2-5 min) with 200 μL 30 mM CHA-IHP. After incubation the system was washed 4× and 15 μL RBC were used for measurement of the Hb-O₂ dissociation curve at 37° C. Observed P₅₀=58.2 Incubation Time: 2-5 min at 37° C. All Experiments were conducted with whole blood.

FIG. 13 depicts oxygen dissociation curves of whole blood treated with a solution of Cyclohexylammonium-Inositol Hexaphosphate (CHA-IHP).

A: Control (25 μL WHOLE BLOOD). Observed P₅₀=23.5

C: 75 μL Whole blood incubated (2-5 min) with 200 μL 30 mM CHA-IHP. After incubation the system was washed 4×. Whole blood Cell Pellet was stored for 48 hrs at 4-8° C. and 15 μL RBC were used for measurement of the Hb-O₂ dissociation curve at 37° C. Observed P₅₀=50.5 Incubation Time: 2-5 min at 37° C. All Experiments were conducted with whole blood.

FIG. 14 depicts oxygen dissociation curves of whole blood treated with a solution of Cyclohexylammonium-Inositol Hexaphosphate (CHA-IHP).

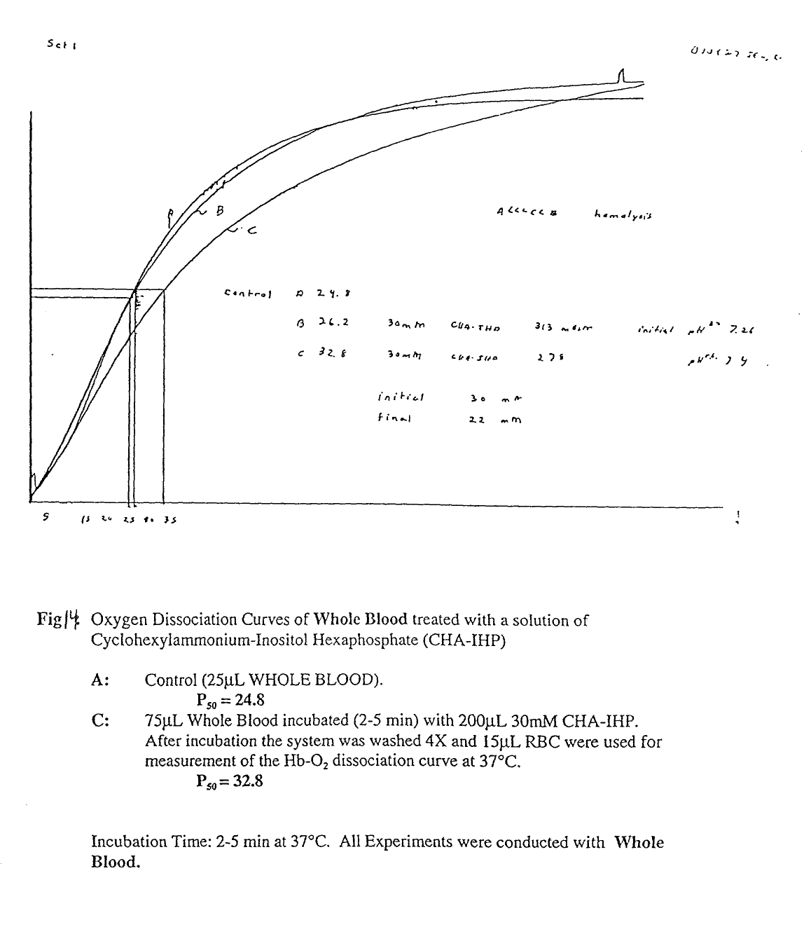

A: Control (25 μL WHOLE BLOOD). Observed P₅₀=24.8

C: 75 μL Whole blood incubated (2-5 min) with 200 μL 30 mM CHA-IHP. After incubation the system was washed 4× and 15 μL RBC were used for measurement of the Hb-O₂ dissociation curve at 37° C. Observed P₅₀=32.8 Incubation Time: 2-5 min at 37° C. All Experiments were conducted with whole blood.

### DETAILED DESCRIPTION OF THE INVENTION

I. Overview

The process of allosterically modifying hemoglobin towards a low oxygen affinity state in whole blood could be used in a wide variety of applications including in treatments for ischemia, heart disease, complications associated with angioplasty, wound healing, radiation therapy of cancer, adult respiratory distress syndrome (ARDS), etc., in extending the shelf-life of blood or restoring the oxygen carrying capacity of out-dated blood, and as sensitizers for x-ray irradiation in cancer therapy, as well as in many other applications.

This invention is related to the use of allosteric hemoglobin modifier compounds in red blood cell suspensions, e.g., in whole blood. Serum albumin, which is the most abundant protein in blood plasma, has been identified as inhibiting the allosteric effects of clofibric acid, bezafibrate, and L3,5/L3,4,5. The precise nature of this inhibition is not fully understood, but appears to be related to these compounds binding to the serum albumin. In contrast, the subject compounds have been found to be relatively unaffected by the presence of serum albumin. Ligands for the allosteric site of hemoglobin that are not adversely effected by serum albumin represent particularly good candidates for drug applications, since the performance of the drug will not be frustrated by the presence of serum albumin present in a patient's blood.

This invention relates to the incorporation of a wide variety of therapeutically useful substances into mammalian red blood cells (RBCs), which could not previously be accomplished without unacceptable losses of RBC contents and/or integrity. In particular, the compounds and methods of the present invention make possible the introduction or incorporation into RBCs of anionic agents, such as DNA, RNA, chemotherapeutic agents, and antibiotic agents. These and other water-soluble substances may be used for a desired slow continuous delivery or targeted delivery when the treated and purified RBC carrier is later injected in vivo. The particular anion or polyanion to be selected can be based on whether an allosteric effector of hemoglobin would be desirable for a particular treatment.

The present invention provides a novel method for increasing the oxygen-carrying capacity of erythrocytes. In accordance with the method of the present invention, the IHP combines with hemoglobin in a stable way, and shifts its oxygen releasing capacity. Erythrocytes with IHP-hemoglobin can release more oxygen per molecule than hemoglobin alone, and thus more oxygen is available to diffuse into tissues for each unit of blood that circulates. IHP is preferably added to red blood cells in vitro or ex vivo, as it appears that it is toxic to animals under certain circumstances.

Another advantage of IHP-treated red blood cells is that they show the Bohr effect in circulation and when stored. Normal red blood cells that have been stored do not regain their maximum oxygen carrying capacity in circulation for approximately 24 hours. This is because the DPG present in normal red blood cells is degraded by native enzymes, e.g., phosphatases, during storage and must be replaced by the body after transfusion. In contrast, red blood cells treated according to the present invention retain their maximum oxygen carrying capacity during storage and therefore can deliver oxygen to the tissues in response to demand immediately after transfusion into a human or animal because there are no native enzymes in erythrocytes which degrade IHP.

IHP-treated RBCs may be used in the treatment of acute and chronic conditions, including, but not limited to, hospitalized patients, cardiovascular operations, chronic anemia, anemia following major surgery, coronary infarction and associated problems, chronic pulmonary disease, cardiovascular patients, autologous transfusions, as an enhancement to packed red blood cells transfusion (hemorrhage, traumatic injury, or surgery) congestive heart failure, myocardial infarction (heart attack), stroke, peripheral vascular disease, intermittent claudication, circulatory shock, hemorrhagic shock, anemia and chronic hypoxia, respiratory alkalemia, metabolic alkalosis, sickle cell anemia, reduced lung capacity caused by pneumonia, surgery, complications associated with angioplasty, pneumonia, trauma, chest puncture, gangrene, anaerobic infections, blood vessel diseases such as diabetes, substitute or complement to treatment with hyperbaric pressure chambers, intra-operative red cell salvage, cardiac inadequacy, anoxia-secondary to chronic indication, organ transplant, carbon monoxide, nitric oxide, and cyanide poisoning.

This invention is related to a method of treating a subject for any one or more of the above diseases comprising the steps of treating red blood cells or whole blood ex vivo with one or more compounds or compositions of the present invention, followed by suitably purifying said red blood cells or whole blood, and administering the thus prepared red blood cells or whole blood to said subject. By ‘suitably purifying’ it is meant a method of washing and separating the red blood cell- or whole blood-allosteric effector suspension and discarding the supernatant until no non-encapsulated allosteric effector can be detected, e.g., as devised by Nicolau et al. in U.S. Pat. No. 5,612,207. Alternatively, a compound of an allosteric effector can be administered directly to a subject if the compound does not have toxic effects in the subject, or at least its beneficial effects predominate over its toxicity in a subject. Toxicity of compounds in a subject could be determined according to methods known in the art.

Treating a human or animal for any one or more of the above disease states is done by transfusing into the human or animal between approximately 0.1 and 6 units (1 unit=500 mL) of IHP-treated blood that has been prepared according to the present invention. In certain cases, blood exchange with IHP-treated blood may be possible. The volume of IHP-treated red blood cells that is administered to the human or animal will depend upon the value of P50 for the IHP-treated RBCs. It is to be understood that the volume of IHP-treated red blood cells that is administered to the patient can vary and still be effective. IHP-treated RBCs are similar to normal red blood cells in every respect except that their P₅₀ value is shifted towards higher partial pressures of O₂. Erythrocytes release oxygen only in response to demand by organs and tissue. Therefore, the compounds, compositions thereof, and methods of the present invention will only restore a normal level of oxygenation to healthy tissue, avoiding the cellular damage that is associated with an over-abundance of oxygen.

Because the compounds, compositions, and methods of the present invention are capable of allosterically modifying hemoglobin to favor the low oxygen affinity “T” state (i.e., right shifting the equilibrium curve), RBC's treated with the compounds of the present invention will be useful in treating a variety of disease states in mammals, including humans, wherein tissues suffer from low oxygen tension, such as cancer and ischemia. Furthermore, as disclosed by Hirst et al. (Radiat. Res., Vol. 112, (1987), pp. 164), decreasing the oxygen affinity of hemoglobin in circulating blood has been shown to be beneficial in the radiotherapy of tumors. RBC's or whole blood treated with the compounds of the present invention may be administered to patients in whom the affinity of hemoglobin for oxygen is abnormally high. For example, certain hemoglobinopathies, certain respiratory distress syndromes, e.g., respiratory distress syndromes in new born infants aggravated by high fetal hemoglobin levels, and conditions in which the availability of hemoglobin/oxygen to the tissues is decreased (e.g., in ischemic conditions such as peripheral vascular disease, coronary occlusion, cerebral vascular accidents, or tissue transplant). The compounds and compositions may also be used to inhibit platelet aggregation, antithrombotic purposes, and wound healing.

Additionally, the compounds and compositions of the present invention can be added to whole blood or packed cells preferably at the time of storage or at the time of transfusion in order to facilitate the dissociation of oxygen from hemoglobin and improve the oxygen delivering capability of the blood. When blood is stored, the hemoglobin in the blood tends to increase its affinity for oxygen by losing 2,3-diphosphoglycerides. As described above, the compounds and compositions of this invention are capable of reversing and/or preventing the functional abnormality of hemoglobin observed when whole blood or packed cells are stored. The compounds and compositions may be added to whole blood or red blood cell fractions in a closed system using an appropriate reservoir in which the compound or composition is placed prior to storage or which is present in the anticoagulating solution in the blood collecting bag.

Administration to a patient can be achieved by intravenous or intraperitoneal injection where the dose of treated red blood cells or whole blood and the dosing regiment is varied according to individual's sensitivity and the type of disease state being treated.

Solid tumors are oxygen deficient masses. The compounds, compositions and methods of this invention may be exploited to cause more oxygen to be delivered to tumors, increasing radical formation and thereby increasing tumor killing during radiation. In this context, such IHP-treated blood will only be used in conjunction with radiotherapy.

The compounds, compositions and methods of this invention may be exploited to cause more oxygen to be delivered at low blood flow and low temperatures, providing the ability to decrease or prevent the cellular damage, e.g., myocardial or neuronal, typically associated with these conditions.

The compounds, compositions and methods of this invention may be exploited to decrease the number of red blood cells required for treating hemorrhagic shock by increasing the efficiency with which they deliver oxygen.

Damaged tissues heal faster when there is better blood flow and increased oxygen tension. Therefore, the compounds, compositions and methods of this invention may be exploited to speed wound healing. Furthermore, by increasing oxygen delivery to wounded tissue, the compounds, compositions and methods of this invention may play a role in the destruction of infection causing bacteria at a wound.

The compounds, compositions and methods of this invention may be effective in enhancing the delivery oxygen to the brain, especially before complete occlusion and reperfusion injuries occur due to free radical formation. Furthermore, the compounds, compositions and methods of this invention of this invention should reduce the expansion of arterioles under both hypoxic and hypotensive conditions.

The compounds, compositions and methods of this invention of this invention should be capable of increasing oxygen delivery to blocked arteries and surrounding muscles and tissues, thereby relieving the distress of angina attacks.

Acute respiratory disease syndrome (ARDS) is characterized by interstitial and/or alveolar edema and hemorrhage as well as perivascular lung edema associated with the hyaline membrane, proliferation of collagen fibers, and swollen epithelium with increased pinocytosis. The enhanced oxygen delivering capacity provided to RBCs by the compounds, compositions and methods of this invention may be effective in the treatment and prevention of ARDS by militating against lower than normal oxygen delivery to the lungs.

There are several aspects of cardiac bypass surgery that make attractive the use of compounds or compositions or methods of the present invention. First, the compounds and compositions of the present invention may be effective as neuroprotective agents. After cardiac bypass surgery, up to 50-70% of patients show some signs of cerebral ischemia based on tests of cognitive function. Up to 5% of these patients have evidence of stroke. Second, cardioplegia is the process of stopping the heart and protecting the heart from ischemia during heart surgery. Cardioplegia is performed by perfusing the coronary vessels with solutions of potassium chloride and bathing the heart in ice water. However, blood cardioplegia is also used. This is where potassium chloride is dissolved in blood instead of salt water. During surgery the heart is deprived of oxygen and the cold temperature helps slow down metabolism. Periodically during this process, the heart is perfused with the cardioplegia solution to wash out metabolites and reactive species. Cooling the blood increases the oxygen affinity of its hemoglobin, thus making oxygen unloading less efficient. However, treatment of blood cardioplegia with compounds or compositions of the present invention will counteract the effects of cold on oxygen affinity and make oxygen release to the ischemic myocardium more efficient, possibly improving cardiac function after the heart begins to beat again. Third, during bypass surgery the patient's blood is diluted for the process of pump prime. This hemodilution is essentially acute anemia. Because the compounds and compositions of the present invention make oxygen transport more efficient, their use during hemodilution (whether in bypass surgery or other surgeries, such as orthopedic or vascular) would enhance oxygenation of the tissues in an otherwise compromised condition. Additionally, the compounds and methods of the present invention will also find use in patients undergoing angioplasty, who may experience acute ischemic insult, e.g., due to the dye(s) used in this procedure.

Additionally, microvascular insufficiency has been proposed by a number of investigators as a possible cause of diabetic neuropathy. The interest in microvascular derangement in diabetic neuropathic patients has arisen from studies suggesting that absolute or relative ischemia may exist in the nerves of diabetic subjects due to altered function of the endo- and/or epineurial blood vessels. Histopathologic studies have shown the presence of different degrees of endoneurial and epineurial microvasculopathy, mainly thickening of blood vessel wall or occlusion. A number of functional disturbances have also been demonstrated in the microvasculature of the nerves of diabetic subjects. Studies have demonstrated decreased neural blood flow, increased vascular resistance, decreased pO₂ and altered vascular permeability characteristics such as a loss of the anionic charge barrier and decreased charge selectivity. Abnormalities of cutaneous blood flow correlate with neuropathy, suggesting that there is a clinical counterpart to the microvascular insufficiency that may prove to be a simple non-invasive test of nerve fiber dysfunction. Accordingly, patients suffering from diabetic neuropathies and/or other neurodegenerative disorders will likely benefit from treatment based on the compounds and methods of the present invention.

RBC's or whole blood treated with the compounds of the present invention may be used to enhance oxygen delivery in any organism, e.g., fish, that use a hemoglobin with an allosteric binding site.

II. Definitions

For convenience, certain terms employed in the specification, examples, and appended claims are collected here. As used throughout this specification and the claims, the following terms have the following meanings:

The term “hemoglobin” includes all naturally- and non-naturally-occurring hemoglobin.

The term “hemoglobin preparation” includes hemoglobin in a physiologically compatible carrier or lyophilized hemoglobin reconstituted with a physiologically compatible carrier, but does not include whole blood, red blood cells or packed red blood cells.

The term “toxic” refers to a property where the deleterious effects are greater than the beneficial effects.

The term “nontoxic” refers to a property where the beneficial effects are greater than the deleterious effects.

The term “whole blood” refers to blood containing all its natural constituents, components, or elements or a substantial amount of the natural constituents, components, or elements. For example, it is envisioned that some components may be removed by the purification process before administering the blood to a subject.

“Purified”, “purification process”, and “purify” all refer to a process or state of removing one or more compounds of the present invention from the red blood cells or whole blood such that when administered to a subject the red blood cells or whole blood is nontoxic.

“Non-naturally-occurring hemoglobin” includes synthetic hemoglobin having an amino-acid sequence different from the amino-acid sequence of hemoglobin naturally existing within a cell, and chemically-modified hemoglobin. Such non-naturally-occurring mutant hemoglobin is not limited by its method of preparation, but is typically produced using one or more of several techniques known in the art, including, for example, recombinant DNA technology, transgenic DNA technology, protein synthesis, and other mutation-inducing methods.

“Chemically-modified hemoglobin” is a natural or non-natural hemoglobin molecule which is bonded to another chemical moiety. For example, a hemoglobin molecule can be bonded to pyridoxal-5′-phosphate, or other oxygen-affinity-modifying moiety to change the oxygen-binding characteristics of the hemoglobin molecule, to crosslinking agents to form crosslinked or polymerized hemoglobin, or to conjugating agents to form conjugated hemoglobin.

“Oxygen affinity” means the strength of binding of oxygen to a hemoglobin molecule. High oxygen affinity means hemoglobin does not readily release its bound oxygen molecules. The P50 is a measure of oxygen affinity.

“Cooperativity” refers to the sigmoidal oxygen-binding curve of hemoglobin, i.e., the binding of the first oxygen to one subunit within the tetrameric hemoglobin molecule enhances the binding of oxygen molecules to other unligated subunits. It is conveniently measured by the Hill coefficient (n[max]). For Hb A, n[max]=3.0.

The term “treatment” is intended to encompass also prophylaxis, therapy and cure.

“Ischemia” means a temporary or prolonged lack or reduction of oxygen supply to an organ or skeletal tissue. Ischemia can be induced when an organ is transplanted, or by conditions such as septic shock and sickle cell anemia. p “Skeletal tissue” means the substance of an organic body of a skeletal organism consisting of cells and intercellular material, including but not limited to epithelium, the connective tissues (including blood, bone and cartilage), muscle tissue, and nerve tissue.

“Ischemic insult” means damage to an organ or skeletal tissue caused by ischemia.

“Subject” means any living organism, including humans, and mammals.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

As used herein, the term “surgery” refers to the treatment of diseases, injuries, and deformities by manual or operative methods. Common surgical procedures include, but are not limited to, abdominal, aural, bench, cardiac, cineplastic, conservative, cosmetic, cytoreductive, dental, dentofacial, general, major, minor, Moh's, open heart, organ transplantation, orthopedic, plastic, psychiatric, radical, reconstructive, sonic, stereotactic, structural, thoracic, and veterinary surgery. The method of the present invention is suitable for patients that are to undergo any type of surgery dealing with any portion of the body, including but not limited to those described above, as well as any type of any general, major, minor, or minimal invasive surgery.

“Minimally invasive surgery” involves puncture or incision of the skin, or insertion of an instrument or foreign material into the body. Non-limiting examples of minimal invasive surgery include arterial or venous catheterization, transurethral resection, endoscopy (e.g., laparoscopy, bronchoscopy, uroscopy, pharyngoscopy, cystoscopy, hysteroscopy, gastroscopy, coloscopy, colposcopy, celioscopy, sigmoidoscopy, and orthoscopy), and angioplasty (e.g., balloon angioplasty, laser angioplasty, and percutaneous transluminal angioplasty).

The term “ED₅₀” means the dose of a drug that produces 50% of its maximum response or effect. Alternatively, the dose that produces a pre-determined response in 50% of test subjects or preparations.

The term “LD₅₀” means the dose of a drug that is lethal in 50% of test subjects.

The term “therapeutic index” refers to the therapeutic index of a drug defined as LD₅₀/ED₅₀.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “structure-activity relationship (SAR)” refers to the way in which altering the molecular structure of drugs alters their interaction with a receptor, enzyme, etc.

The term “ammonium cation” refers to the structure below:

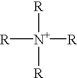

wherein R represents independently for each occurrence H or a substituted or unsubstituted aliphatic group. An “aliphatic ammonium cation” refers to the above structure when at least one R is an aliphatic group. A “quaternary ammomium cation” refers to the above structure when all four occurrences of R independently represent aliphatic groups. R can be the same for two or more occurrences, or different for all four.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulthydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

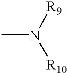

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

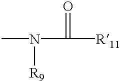

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

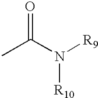

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S—alkyl, —S—alkenyl, —S—alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

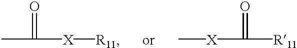

wherein X is a bond or represents an oxygen or a sulfur, and R₁₁ represents a hydrogen, an alkyl, an alkenyl, —(CH2)ₘ—R₈ or a pharmaceutically acceptable salt, R′_{1 1} represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —-alkynyl, —O—(CH₂)ₘ—R₈, where m and R₈ are described above.

The term “sulfonate” is art recognized and includes a moiety that can be represented by the general formula:

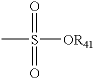

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

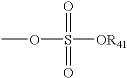

in which R₄₁ is as defined above.

The term “sulfonamido” is art recognized and includes a moiety that can be represented by the general formula:

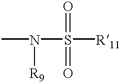

in which R₉ and R′₁₁ are as defined above.

The term “sulfamoyl” is art-recognized and includes a moiety that can be represented by the general formula:

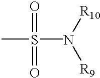

in which R₉ and R₁₀ are as defined above.

The term “sulfonyl”, as used herein, refers to a moiety that can be represented by the general formula:

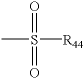

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

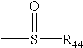

in which R₄₄ is selected from the group consisting of hydrogen, alkyl, alkenyl, alkynyl, cycloalkyl, heterocyclyl, aralkyl, or aryl.

A “phosphoryl” can in general be represented by the formula:

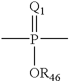

wherein Q₁ represented S or O, and R₄₆ represents hydrogen, a lower alkyl or an aryl. When used to substitute, e.g., an alkyl, the phosphoryl group of the phosphorylalkyl can be represented by the general formula:

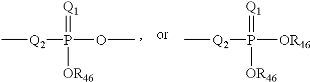

wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P.G.M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

III. Compounds of the Invention

Several years ago, it was discovered that the antilipidemic drug clofibric acid lowered the oxygen affinity of hemoglobin solutions (Abraham et al., J. Med. Chem. 25, 1015 (1982), and Abraham et al., Proc. Natl. Acad. Sci. USA 80, 324 (1983)). Bezafibrate, another antilipidemic drug, was later found to be much more effective in lowering the oxygen affinity of hemoglobin solutions and suspensions of fresh, intact red cells (Perutz et al., Lancet, 881, Oct. 15, 1983). Subsequently, X-ray crystallographic studies have demonstrated that clofibric acid and bezafibrate bind to the same sites in the central water cavity of deoxyhemoglobin, and that one bezafibrate molecule will span the sites occupied by two clofibric acid molecules. Bezafibrate and clofibric acid act by stabilizing the deoxy structure of hemoglobin, shifting the allosteric equilibrium toward the low affinity deoxy form. Bezafibrate and clofibric acid do not bind in any specific manner to either oxy- or carbonmonoxyhemoglobin.

In later investigations, a series of urea derivatives [2-[4-[[(arylamino)carbonyl]-amino]phenoxy]-2-methylpropionic acids] was discovered that has greater allosteric potency than bezafibrate at stabilizing the deoxy structure of hemoglobin and shifting the allosteric equilibrium toward the low oxygen affinity form (Lalezari, Proc. Natl. Acad. Sci. USA 85, 6117 (1988)).

It has been determined that certain allosteric hemoglobin modifier compounds are hydrophobic molecules that can be bound to the body's neutral fat deposits and lipophilic receptors sites, thus lowering their potency due to a decreased concentration in RBCs. Administration of a hydrophobic compound, such as a mixture of anesthetic molecules, will saturate the body's neutral fat deposits and lipophilic receptor sites, and thereby increase the concentration of this type of allosteric modifiers in RBCs, where higher concentrations of effector will increase its ability to interact with hemoglobin, causing delivery of more oxygen.

Ligands for the allosteric site of hemoglobin, also known as allosteric effectors of hemoglobin, include 2,3-diphosphoglycerate (DPG), inositol hexakisphosphate (IHP), bezafibrate (Bzf), LR16 and L35 (two recently synthesized derivatives of Bzf), and pyridoxal phosphate. Additionally, hemoglobin's affinity for oxygen can be modulated through electrostatic interactions with chloride and/or organophosphate anions present in RBCs. These effectors, which bind preferentially to the deoxy-Hb tetramers at a distance from the heme groups, play a major role in the adaptation of the respiratory properties of hemoglobin to either allometric-dependent oxygen needs or to various hypoxic environments. Additionally, protons and carbon dioxide are physiological regulators for the oxygen affinity of hemoglobin. The heterotropic allosteric interaction between the non-heme ligands and oxygen, collectively called the Bohr effect, facilitates not only the transport of oxygen but also the exchange of carbon dioxide.

The present invention relates to compositions, and methods of use thereof, consisting essentially of an ammonium cation (preferably water-soluble), and an anionic ligand for the zallosteric site of hemoglobin, e.g., inositol hexaphosphate (IHP). In certain embodiments, the quaternary ammonium cation is represented by the general formula N(R)₄, wherein R is, independently for each occurrence, H or an aliphatic group, preferably an alkyl, more preferably a lower (C1-C6) alkyl, and even more preferably a C1-C10 cyclic alkyl. In certain preferred embodiments, the quaternary ammonium cation is preferably derived from cyclic organic bases.

In certain embodiments, the present invention is related to compounds, and compositions thereof, which deliver into erythrocytes allosteric modifiers of hemoglobin ex vivo. Additionally, the invention is directed to the use of the compounds or compositions thereof that are effective in delivering into erythrocytes allosteric modifiers of hemoglobin, lowering the oxygen affinity state in red blood cell suspensions and whole blood. It is an object of this invention to provide methods for delivering into erythrocytes allosteric modifiers of hemoglobin in whole blood, utilizing compounds or compositions thereof that do not lose their effectiveness in the presence of normal concentrations of the remaining components of whole blood.

In certain embodiments, the present invention is related to a method of treating red blood cells or whole blood ex vivo with one or more nontoxic compounds or compositions of the present invention, suitably purifying said red blood cells or whole blood, and administering said purified red blood cells or whole blood to a subject for any treatment where an increase in oxygen delivery by hemoglobin would be a benefit.

In part, the present invention is directed toward the design of water-soluble membrane compatible molecules comprising ammonium cationic moieties, e.g., lipophilic quaternary ammonium groups. These molecules form complexes with anionic molecules, e.g., ligands for the allosteric site of hemoglobin; such complexes are useful for the deliver of said anionic molecules into the cytoplasm of mammalian cells, e.g., erythrocytes.

The ammonium group of the cationic component of the compounds of the present invention is particularly well suited for interaction with the phosphate residues of IHP and congeners thereof because of the coulombic interactions, i.e., the attraction between opposite charges, that can be established between the two moieties. We report here the use of ammonium salts for the efficient delivery into mammalian erythrocytes of phosphate-containing ligands for the allosteric site of hemoglobin. Our data demonstrate the usefulness, convenience, and versatility of ammonium salts for delivery of small anionic molecules into the cytoplasm of mammalian cells.

In certain embodiments, the compounds of the present invention are represented by generalized structure 1:

n C^{\mathring{+}}A^{n\mathring{-}} 1

wherein

C⁺ represents independently for each occurrence an aliphatic ammonium cation, an alkali metal cation, or an alkaline earth cation; provided that at least one instance of C⁺ represents an aliphatic ammonium cation;

Aⁿ⁻ represents an anionic ligand for a mammalian cellular receptor; and

n is an integer in the range 1 to 12 inclusive.

In certain embodiments, the compounds of the present invention are represented by generalized structure 1, and the attendant definitions, wherein an instance of C⁺ that represents an ammonium ion is selected independently for each occurrence from the group consisting of C₁-C₆ alkyl ammonium ions and C₃-C₆ cycloalkyl ammonium ions.

In certain embodiments, the compounds of the present invention are represented by generalized structure 1, and the attendant definitions, wherein an instance of C⁺ that represents an ammonium ion is selected independently for each occurrence from the group consisting of C₃-C₆ cycloalkyl ammonium ions.

In certain embodiments, the compounds of the present invention are represented by generalized structure 1, and the attendant definitions, wherein an instance of C⁺ that represents an ammonium ion is selected independently for each occurrence from the group consisting of cyclohexyl ammonium ions.

In certain embodiments, the compounds of the present invention are represented by generalized structure 1, and the attendant definitions, wherein Aⁿ⁻ is a ligand for the allosteric site of hemoglobin.

In certain embodiments, the compounds of the present invention are represented by generalized structure 1, and the attendant definitions, wherein Aⁿ⁻ is a phosphorylated inositol or a phosphorylated glyceric acid.

In certain embodiments, the compounds of the present invention are represented by generalized structure 1, and the attendant definitions, wherein Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by generalized structure 1, and the attendant definitions, wherein Aⁿ⁻ is IHP or 2,3-DPG.

In certain embodiments, the compounds of the present invention are represented by generalized structure 1, and the attendant definitions, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid.

In certain embodiments, the compounds of the present invention are represented by generalized structure 1, and the attendant definitions, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is IHP or 2,3-DPG.

In certain embodiments, the compounds of the present invention are represented by generalized structure 1, and the attendant definitions, wherein an instance of C⁺ that represents an ammonium ion is selected independently for each occurrence from the group consisting of C₃-C₆ cycloalkyl ammonium ions; and Aⁿ⁻ is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of C₃-C₆ cycloalkyl ammonium ions; and A^{n'1} is a phosphorylated inositol or a phosphorylated glyceric acid.

In certain embodiments, the compounds of the present invention are represented by generalized structure 1, and the attendant definitions, wherein an instance of C⁺ that represents an ammonium ion is selected independently for each occurrence from the group consisting of C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of C₃-C₆ cycloalkyl ammonium ions; and Aⁿ⁻ is IHP or 2,3-DPG.

In certain embodiments, the compounds of the present invention are represented by generalized structure 1, and the attendant definitions, wherein an instance of C⁺ that represents an ammonium ion is selected independently for each occurrence from the group consisting of cyclohexyl ammonium ions; and Aⁿ⁻ is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of cyclohexyl ammonium ions; and Aⁿ⁻ is a phosphorylated inositol or a phosphorylated glyceric acid.

In certain embodiments, the compounds of the present invention are represented by generalized structure 1, and the attendant definitions, wherein an instance of C⁺ that represents an ammonium ion is selected independently for each occurrence from the group consisting of cyclohexyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by generalized structure 1, and the attendant definitions, wherein an instance of C⁺ that represents an ammonium ion is selected independently for each occurrence from the group consisting of cyclohexyl ammonium ions; and Aⁿ⁻ is IHP or 2,3-DPG.

In certain embodiments, the present invention relates to a pharmaceutical composition, comprising a nontoxic compound of the present invention; and a pharmaceutically acceptable excipient.

IV. Methods of the Invention

In certain embodiments, the method of the present invention comprises the step of administering to a subject red blood cells or whole blood that has previously been treated with a compound or composition of the present invention ex vivo and wherein said red blood cells or whole blood has been subsequently suitably purified such that when said red blood cells or whole blood is administered to a subject it is nontoxic to said subject.

In certain embodiments, the method of the present invention comprises the step of administering to a subject red blood cells or whole blood that has previously been treated ex vivo with a compound or composition of the present invention wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject experiencing ischemia red blood cells or whole blood that has previously been treated ex vivo with a compound or composition of the present invention and wherein said red blood cells or whole blood has subsequently been suitably purified such that when said red blood cells or whole blood is administered to a subject it is nontoxic to said subject.

In certain embodiments, the method of the present invention comprises the step of administering to a subject experiencing ischemia red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject experiencing cardiac arrhythmia red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject experiencing cardiac arrhythmia red blood cells or whole blood that has previously been treated ex vivo with a compound or composition of the present invention wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject experiencing a heart attack red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject experiencing a heart attack red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject experiencing a stroke red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject experiencing a stroke red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject experiencing hypoxia red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject experiencing hypoxia red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject afflicted with sickle cell anemia red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject afflicted with sickle cell anemia red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject suffering from hypotension red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject suffering from hypotension red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject suffering from arteriosclerosis red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject suffering from arteriosclerosis red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject suffering from altitude sickness red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject suffering from altitude sickness red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of administering to a subject suffering from diabetes red blood cells or whole blood that has previously been treated ex vivo with a compound or composition of the present invention, and wherein said red blood cells or whole blood has subsequently been suitably purified such that when said red blood cells or whole blood is administered to a subject it is nontoxic to said subject.

In certain embodiments, the method of the present invention comprises the step of administering to a subject suffering from diabetes red blood cells or whole blood that has previously been treated ex vivo with a compound or composition of the present invention, wherein said red blood cells or whole blood has subsequently been suitably purified such that when said red blood cells or whole blood is administered to a subject it is nontoxic to said subject, and wherein said administration is intravenous.

In certain embodiments, the method of the present invention comprises the step of adding to mammalian blood a compound or composition of the present invention.

In certain embodiments, the method of the present invention comprises the step of adding to plasma comprising mammalian erythrocytes a compound or composition of the present invention.

V. Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. A natural requirement for any pharmaceutically acceptable composition is that it comprises a nontoxic compound of the present invention. We are aware that many of the modem drugs of great benefit have started out as toxic substances. Ongoing research in our laboratories is directed towards nontoxic compounds of ammonium salts and anionic allosteric effectors. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (I) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

VI. Administration of the Compounds of the Present Invention

In another aspect, the current invention provides methods of administering to a subject pharmaceutical compositions comprised of a nontoxic ammonium salt of an anionic allosteric effector. Many techniques currently exist for delivering drugs or other medicaments to body tissue. These include, among possible others, oral administration, injection directly into body tissue such as through an intramuscular injection or the like, topical or transcutaneous administration where the drug is passively absorbed, or caused to pass, into or across the skin or other surface tissue and intravenous administration which involves introducing a selected drug directly into the blood stream. Techniques and formulations generally may be found in Remmington's Pharmaceutical Sciences, Meade Publishing Co., Easton, Pa.

Exemplification

The invention now being generally described, it will be more readily understood by reference to the following examples, which are included merely for purposes of illustration of certain aspects and embodiments of the present invention, and are not intended to limit the invention.

### EXAMPLE 1

This example shows that ammonium salts of 2,3 Diphospho-D-Glyceric Acid (DPG) and Inositol Hexaphosphate (IHP) improves the dissociation of oxygen from hemoglobin following incubation with whole blood.

A. Effectors

2,3 Diphospho-D-Glyceric Acid (Pentacyclohexyclammonium salt) (PCHA-DPG)

C₃H₈O₁₀P₂.5C₆H₁₃N

F.W.: 761.9

2,3 Diphospho-D-Glyceric Acid (Pentasodium salt) (PNa-DPG)

C₃H₈O₁₀P₂Na₅

F.W.: 375.9

Cyclohexylamine (CHA)

C₆H₁₃N

F.W.: 99.18

Inositol Hexaphosphate (IHP)

myo-inositol hexakis (dehydrogen Phosphate)

CHA-IHP was prepared by titration of acidic IHP with alkaline CHA to a pH of 7.1-7.4.

B. Blood Preparations

Whole blood was collected from one subject. The blood was stored in a Vacutainer with Solution A (ACD) and stored at 4-8° C.

To isolate red blood cells, whole blood (3 mL) was placed on top of test tube containing 9 mL of Histopaque 1119 (Sigma Diagnostics Inc.) and 1 mL of Saline buffer. Following centrifugation the supernatant and buffy coat were removed and the pellet containing RBCs were washed three times in 10 mL HBS.

C. Buffers

HBS=HEPES Buffered Saline,

HBS was used as the standard buffer for experiments. HBS 7.42 (r.t.) was ideal to keep pH of experiments at 7.28-7.32 (37° C.).

20 mM HEPES

130 mM Sodium Chloride

HEPES, (N-[2-hydroxyethyl]piperazine-N′-[2-ethanesulfonic acid])

C₈H₁₈N₂O₄S

F.W.=238.3

pKₐ=7.5

pH: 6.8-8.2

CAS#7365-45-9

HBS+HBS

20 μL Bovine Serum Albumin (BSA) per 5 mL HBS (TCS Medical Products Company)

15 μL Antifoaming per 5 mL HBS (TCS Medical Products Company)

pH: 7.2-7.4

Osmolarity: 290-320 mOsM

HBS.BSA 5 mL HBS Plus 20 μL BSA saline, 0.9% Sodium Chlorida, Injection USP

Each 100 mL contains:

| | |
|---|---|
| 900 mg | NaCl |
| 154 mEq/L | Sodium |
| 154 mEq/L | Chloride |

pH: 5.0

Osmolarity: 308 mOsM

BIS-TRIS buffered saline, (bis[2-Hydroxyethyl]minotris[hydroxymethyl]methane), (Sigma).

20 mM Bis-Tris

140 mM Sodium Chloride

pH: 7.45

Osmolarity: 294 mOsM

D. Procedures

Preparation of Effector Stock: Effector stock was prepared at 100-120 mM (Molal solution) using water or Bis-Tris Buffer. Effector characteristics prior to incubation were:

| | |
|---|---|
| Concentration: | 30 mM |
| Osmolarity: | 170-340 mOsM |
| pH: | 7.1-7.4 (at 37° C.) |

Incubation: Whole blood (75-300 μL) was incubated with 200 μL of effector at 37° C. for 5-10 min. (see Summary of Results below).

Washes: After incubation of whole blood with/without effector, blood cells were washed four times with Saline buffer or HBS (BSA) by centrifugal pelleting to remove exogenous effector and to evaluate hemolysis. After final centrifugation, pellet was not resuspended.

Blood Oxygen Dissociation Reading: Blood Oxygen Dissociation of samples were determined using a Hemox Analizer Model B (TCS Medical Products Company, New Hope, Pa.) The sample chamber contained:

Control:

2.5-3.0 mL of HBS+

25 μL Whole blood

Effector evaluation:

2.5-3.0 mL of HBS+

10-20 μL Pelleted Blood Cells incubated with Effector.

All readings were made at 36.7-37.2° C. and at pH 7.28-7.32. The P₅₀S were calculated from the Dissociation Curves (see FIGS. 16-23) compared to same day control P₅₀. The results are presented in Table 2.

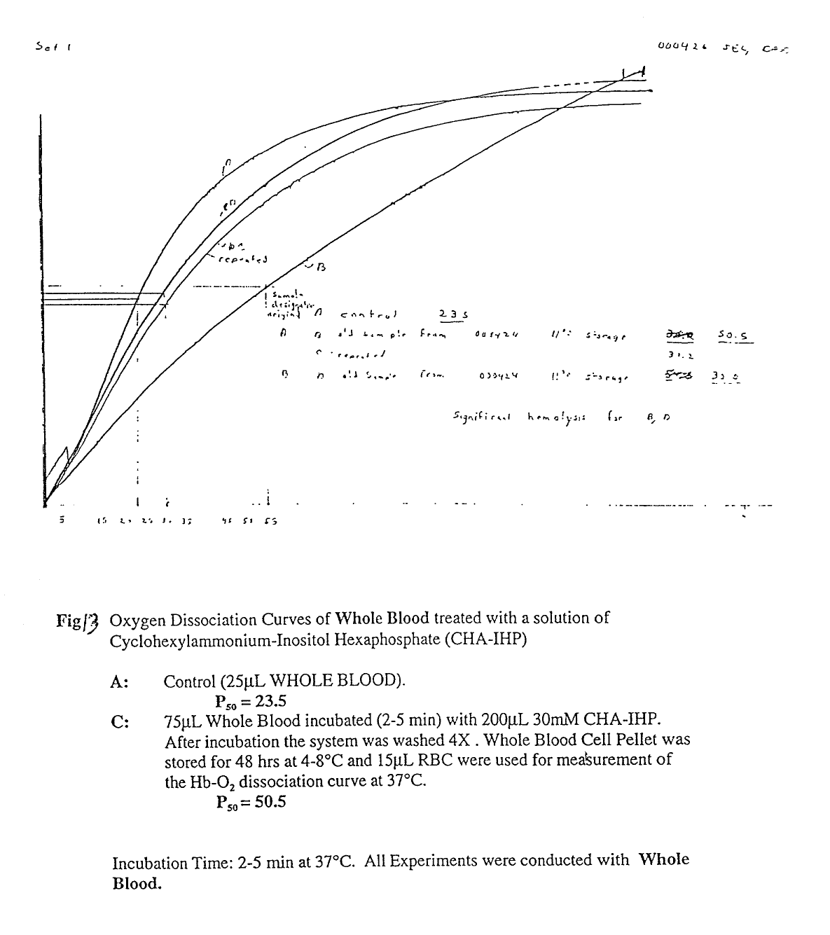

TABLE 1
Summary of Results from Example 1.
P₅₀ of Whole Blood Pre-Incubated with Effector (Low Osmolarity)
(All incubations and measurements at 37 +/− 0.2 C.)

| | P₅₀ | | | | | | | |
|---|---|---|---|---|---|---|---|---|
| | CONTROL | P₅₀ | CONC. | CONC | OSMOL. | | pH | Volume |
| | WB | EFF:WB | EFF | EFF:WB | EFF | pH | EFF: | Ratio |
| EFFECTOR | mmHg | mmHg | mM | mM | mOsM | EFF. | WB | EFF:WB |
| PCHA- | | | | | | | | |
| DPG | | | | | | | | |
| | 37 | 55.5 | 30 | 22 | 205 | | 7.42 | 1:0.375 |
| | 25.5 | 47.3 | 33 | 23 | 185 | 7.9 | | 1:0.375 |
| | 25 | 36 | 30 | 22 | 221 | 7.8 | 7.33 | 1:0.375 |
| | 25 | 40 | 30 | 22 | 209 | 8.94 | 7.98 | 1:0.375 |
| 5Na-DPG | | | | | | | | |
| | 37 | 38.2 | 30 | 22 | 163 | 7.87 | | 1:0.375 |
| | 37 | 37.5 | 30 | 22 | 163 | 7.43 | | 1:0.375 |
| IHP | | | | | | | | |
| | 37 | 38.2 | 30 | 22 | 185 | | 7.3 | 1:0.375 |
| | 28 | 28.6 | 30 | 22 | 108 | | 7.43 | 1:0.375 |
| CHA | | | | | | | | |
| | 26.8 | 28.5 | 30 | 22 | 220 | | 6.23 | 1:0.375 |
| | 26.8 | 26.8 | 30 | 22 | 245 | | 6.75 | 1:0.375 |
| CHA-IHP | | | | | | | | |
| | 26.8 | 42 | 30 | 22 | 220 | | 6.36 | 1:0.375 |
| FIG. | 24.7 | 58.2 | 25 | 14.3 | 171 | | 6.93 | 1:0.375 |
| 12C* FIG. 13 | 23.5 | 50.5 | 25 | 14.3 | 171 | | 6.93 | 1:0.375 |
| B | 24.8 | 32.8 | 30 | 22 | 278 | | 7.4 | 1:0.375 |
| | 30 | 40.7 | 30 | 22 | 123 | | 6.95 | 1:0.375 |
| | 30 | 45.2 | 30 | 22 | 125 | | 7.667 | 1:0.375 |

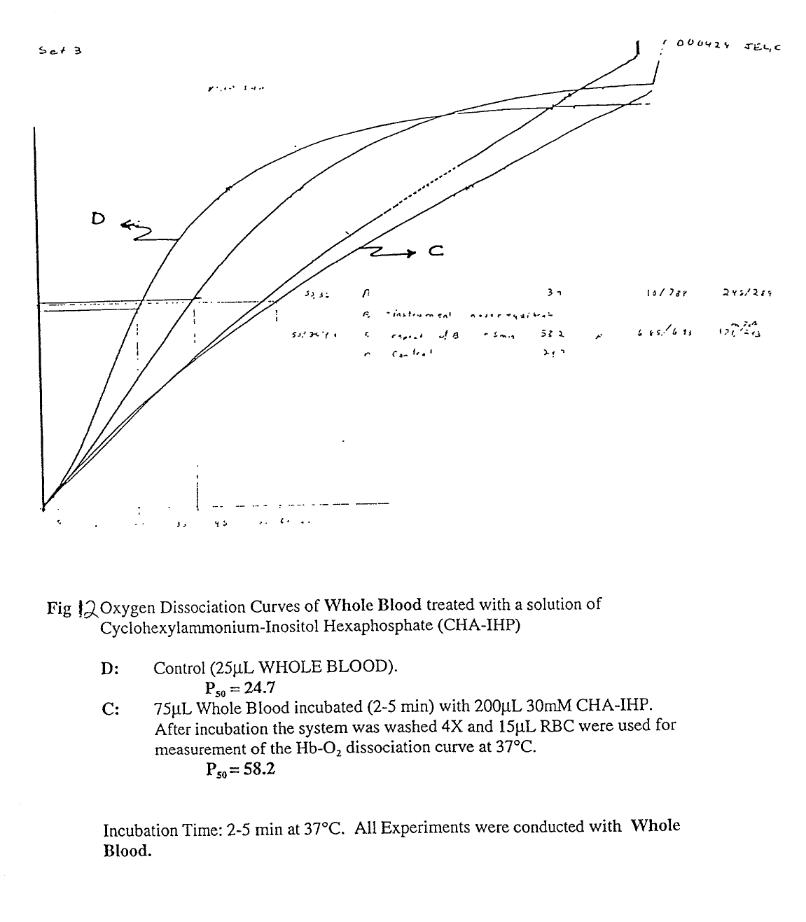

WB = whole blood; EFF = allosteric effector; PCHA-DPG = pentacyclohexylammonium 2,3-diphosphoglyceric acid; 5Na-DPG = penta sodium 2,3-diphosphoglyceric acid; IHP = inositol hexaphosphate; CHA = cyclohexylammonium.
The control value for whole blood's P₅₀ varies due to aging of the blood. Aging is accompanied by the degradation of natural allosteric effectors by native phosphotases.
*=same sample as FIG. 12C, evaluated 48 h later (stored at 4-8 C.).

E. Observations

2,3 Diphospho-D-Glyceric Acid (Pentacyclohexylammonium salt) (PCHA-DPG)

1) The solution is visually clear.

2) There is no aggregation of Red Blood Cells.

3) Increases the P₅₀ of Red Blood Cells

4) Treated-washed RBC pellet maintain higher P50 than the control after resuspended in HBS.

2,3 Diphospho-D-Glyceric Acid (Pentasodium salt) (PNa-DPG)

1) The solution is visually clear.

2) There is no aggregation of Red Blood Cells.

3) No change of the P₅₀ of treated Red Blood Cells vs. control.

Cyclohexylamine (CHA)

1) The solution is visually clear.

2) There is no aggregation of Red Blood Cells.

3) No change of the P₅₀ of treated Red Blood Cells vs. control.

Inositol Hexaphosphate (IHP, Sodium Salt)

1) The solution is visually clear.

2) There is no aggregation of Red Blood Cells.

3) No change of the P₅₀ of treated Red Blood Cells vs. control.

Cyclohexylammonium—Inositol Hexaphosphate (CHA.IHP)

1) The solution is visually clear.

2) There is no aggregation of Red Blood Cells.

3) Increases the P₅₀ of Red Blood Cells

4) Treated-washed RBC pellet maintain higher P₅₀ than the control after resuspended in HBS.

F. Conclusion

Ammonium Salts of DPG and IHP increase the P₅₀ of whole blood in comparison to the sodium salts of these two allosteric effectors at osmolarities less than 280 mOsM.

Incorporation by Reference

All of the patents and publications cited herein are hereby incorporated by reference.

Equivalents

Those skilled in the art will recognize, or be able to ascertain using no more than routine experimentation, many equivalents to the specific embodiments of the invention described herein. Such equivalents are encompassed by the following claims.

We claim:

1. A composition, comprising a salt complex of an aliphatic ammonium cation and an anionic ligand for a mammalian cellular receptor.

2. The composition of claim 1, wherein the anionic ligand is a ligand for the allosteric site of hemoglobin.

3. The composition of claim 2, wherein the anionic ligand is a phosphorylated inositol or a phosphorylated glyceric acid.

4. The composition of claim 3, wherein the anionic ligand is inositol hexaphosphate or 2,3-DPG.

5. The composition of claim 3, wherein the anionic ligand is inositol hexaphosphate.

6. The composition of claim 1, wherein the aliphatic ammonium cation is a lipophilic, water-soluble aliphatic ammonium cation.

7. The composition of claim 6, wherein the aliphatic ammonium cation is a monoalkyl, dialkyl, trialkyl or tetraalkyl ammonium moiety.

8. The composition of claim 7, wherein the aliphatic ammonium cation is a monoalkyl ammonium cation.

9. The composition of claim 2, wherein the aliphatic ammonium cation is a primary ammonium cation.

10. A compound represented by generalized structure 1:
n C^{\mathring{+}}A^{n\mathring{-}} 1
wherein
C⁺ represents independently for each occurrence an aliphatic ammonium cation, an alkali metal cation, or an alkaline earth cation; provided that at least one instance of C⁺ represents an aliphatic ammonium cation;
Aⁿ⁻ represents an anionic ligand for a mammalian cellular receptor; and
n is an integer in the range 1 to 12 inclusive.

11. The compound of claim 10, wherein an instance of C⁺ that represents an ammonium ion is selected independently for each occurrence from the group consisting of C₁-C₆ alkyl ammonium ions and C₃-C₆ cycloalkyl ammonium ions.

12. The compound of claim 10, wherein an instance of C⁺ that represents an ammonium ion is selected independently for each occurrence from the group consisting of C₃-C₆ cycloalkyl ammonium ions.

13. The compound of claim 10, wherein an instance of C⁺ that represents an ammonium ion is selected independently for each occurrence from the group consisting of cyclohexyl ammonium ions.

14. The compound of claim 10, wherein Aⁿ⁻ is a ligand for the allosteric site of hemoglobin.

15. The compound of claim 10, wherein Aⁿ⁻ is a phosphorylated inositol or a phosphorylated glyceric acid.

16. The compound of claim 10, wherein Aⁿ⁻ is a phosphorylated inositol or a phosphorylated glyceric acid, wherein said phosphorylated inositol or phosphorylated glyceric acid is a ligand for the allosteric site of hemoglobin.

17. The compound of claim 10, wherein Aⁿ⁻ is IHP or 2,3-DPG.

18. The compound of claim 10, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a ligand for the allosteric site of hemoglobin.

19. The compound of claim 10, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid.

20. The compound of claim 10, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

21. The compound of claim 10, wherein an instance of C⁺ that represents an ammonium ion is selected independently for each occurrence from the group consisting of C₁-C₆ alkyl ammonium ions and C₃-C₆ cycloalkyl ammonium ions; and Aⁿ⁻ is IHP or 2,3-DPG.

22. The compound of claim 10, wherein an instance of C⁺ that represents an ammonium ion is selected independently for each occurrence from the group consisting of C₃-C₆ cycloalkyl ammonium ions; and Aⁿ⁻ is a ligand for the allosteric site of hemoglobin.

23. The compound of claim 10, wherein an instance of C⁺ that represents an ammonium ion is selected independently for each occurrence from the group consisting of C₃-C₆ cycloalkyl ammonium ions; and Aⁿ⁻ is a phosphorylated inositol or a phosphorylated glyceric acid.

24. The compound of claim 10, wherein an instance of C⁺ that represents an ammonium ion is selected independently for each occurrence from the group consisting of C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

25. The compound of claim 10, wherein an instance of C⁺ that represents an ammonium ion is selected independently for each occurrence from the group consisting of C₃-C₆ cycloalkyl ammonium ions; and Aⁿ⁻ is IHP or 2,3-DPG.

26. The compound of claim 10, wherein an instance of C⁺ that represents an ammonium ion is selected independently for each occurrence from the group consisting of cyclohexyl ammonium ions; and Aⁿ⁻ is a ligand for the allosteric site of hemoglobin.

27. The compound of claim 10, wherein an instance of C⁺ that represents an ammonium ion is selected independently for each occurrence from the group consisting of cyclohexyl ammonium ions; and Aⁿ⁻ is a phosphorylated inositol or a phosphorylated glyceric acid.

28. The compound of claim 10, wherein an instance of C⁺ that represents an ammonium ion is selected independently for each occurrence from the group consisting of cyclohexyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

29. The compound of claim 10, wherein an instance of C⁺ that represents an ammonium ion is selected independently for each occurrence from the group consisting of cyclohexyl ammonium ions; and Aⁿ⁻ is IHP or 2,3-DPG.

30. A method of enhancing oxygen delivery to a tissue or organ of a mammal, comprising the step of administering to said mammal, red blood cells or whole blood previously treated with a composition of claim 2 or a compound of claim 10 and subsequently suitably purified such that when said red blood cells or whole blood is administered to said mammal it is nontoxic.

31. A method of treating a mammal afflicted with anemia, coronary infarction, pulmonary disease, congestive heart failure, diabetes, myocardial infarction, stroke, peripheral vascular disease, intermittent claudication, circulatory shock, hemorrhagic shock, chronic hypoxia, altitude sickness, arteriosclerosis, respiratory alkalemia, metabolic alkalosis, sickle cell anemia, reduced lung capacity, gangrene, anaerobic infections, carbon monoxide poisoning, nitric oxide poisoning, or cyanide poisoning, comprising the step of administering to said mammal red blood cells or whole blood previously treated with a composition of claim 2 or a compound of claim 10 and subsequently suitably purified such that when said red blood cells or whole blood is administered to said mammal it is nontoxic.

32. A method of improving the oxygen delivering capability of mammalian blood, comprising the step of adding to said mammalian blood a composition of claim 2 or a compound of claim 10.

33. A method of incorporating a therapeutically useful substance into mammalian red blood cells, comprising the step of treating said mammalian red blood cells with a composition of claim 1 or a compound of claim 10, wherein said composition or compound comprises said therapeutically useful substance.
